(19) United States
(12) Patent Application Publication
(10) Pub. No.: US 2007/0148672 A1
(43) Pub. Date: Jun. 28, 2007

(54) System and method of facilitating animal propagation

(75) Inventors: Casey Worrell, Harper, TX (US); E. Duane Heady, San Antonio, TX (US)

Correspondence Address: Daniel D. Chapman;Jackson Walker, LLP, Suite 2400, 112 E. Pecan, San Antonio, TX, 78205, US

(73) Assignee: Bull's Eye Solutions, LLC

(21) Appl. No.: 11/607,674

(22) Filed: Dec. 1, 2006

Related U.S. Application Data
(63) Continuation-in-part of application No. 11/293,537, filed on Dec. 2, 2005.

(51) Int. Cl. C12Q 1/68; G06F 19/00

(52) U.S. Cl. 435/6; 702/20

(57) ABSTRACT

The present invention provides a system and method of facilitating animal propagation. In one embodiment, the present invention utilizes data relating to the traits or characteristics of existing animals organized into various cow-bull pairs to predict the degree that those traits or characteristics will be exhibited by the offspring of one or more of the animal pairs. Such data is utilized to calculate the likelihood and/or probability that a pair of animals will produce offspring having one or more desirable characteristics. The present invention allows the user to enter preferences regarding one or more offspring traits. In one embodiment, the user may prioritize one or more traits such that greater weight is given to certain traits during calculation. Traits may also be maximized, minimized or optimized in order to address the needs of each individual user. Results may also be displayed and ranked according to one or more user preferences.

This application is a continuation-in-part of and claims priority from U.S. patent application Ser. No. 11/293,537, filed on Dec. 2, 2005.

### FIELD OF THE INVENTION

The present invention relates generally to animal propagation, and more particularly to a system and method of facilitating animal propagation.

### BACKGROUND OF THE INVENTION

Animal owners have continually wrestled with the inconsistency and uncertainty of known breeding techniques. In many cases, animal owners select animals for breeding based upon visual inspection of their animals. In short, a male and a female are selected based upon one or more observed desirable traits in the hopes that the male and female, if bred to one another, will produce offspring having similar traits. Unfortunately, such techniques are unreliable and time consuming.

As such, there remains a need for a system and method capable of reducing the uncertainty associated with animal breeding.

### SUMMARY OF THE INVENTION

Accordingly, the present invention provides a system and method of facilitating animal propagation. In one embodiment, the present invention utilizes data relating to the traits or characteristics exhibited by the offspring of one or more animals. Such data is utilized to calculate the likelihood and/or probability that a pair of animals will produce offspring having one or more desirable characteristics.

In one embodiment, the calculations made possible by the unique functionality of the present invention are performed with the assistance of a computer or other electronic device. In one embodiment, the present invention provides a web-based software application designed to interact with remote users via a computer network.

Users of the present invention are provided with an attractive graphic user interface through which they may enter animal information into the system. In one embodiment, such information may be categorized and stored according to ranch, herd, or pasture. In one embodiment, the present invention utilizes progeny predictors relating to a plurality of animals.

Upon selection of a potential mating pair, the present invention utilizes progeny predictors associated with each animal to estimate the progeny predictors most likely to be associated with the potential offspring of the potential mating pair. In one embodiment, the calculation performed by the present invention involves taking the average of the progeny predictors or EPDs of a set of potential mating pair. The averaged progeny predictors of the potential mating pair provides an estimation of the likely traits to be exhibited by the offspring of the potential mating pair, should the user decide to breed these animals.

The present invention allows the user to enter preferences regarding offspring traits. In one embodiment, the user may emphasize one or more traits such that greater weight is given during calculation. Traits may also be maximized, minimized, or optimized in order to address the needs of each individual user. Results may also be displayed and ranked according to one or more user preferences.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings; it being understood that the drawings contained herein are not necessarily drawn to scale; wherein:

FIG. 1 is a component diagram of one embodiment of the present invention.

FIG. 2 is a flow chart diagram illustrating one embodiment of the present invention.

FIG. 3 is a flow chart diagram illustrating one embodiment of the present invention.

FIGS. 4-12 are screen shots illustrating the graphic user interface of one embodiment of the present invention.

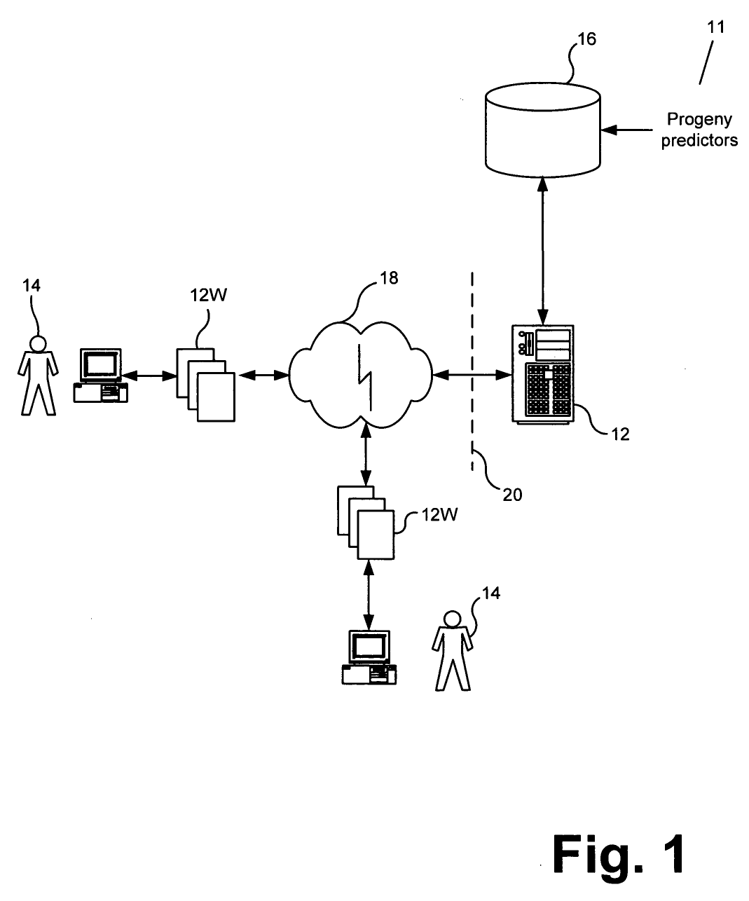

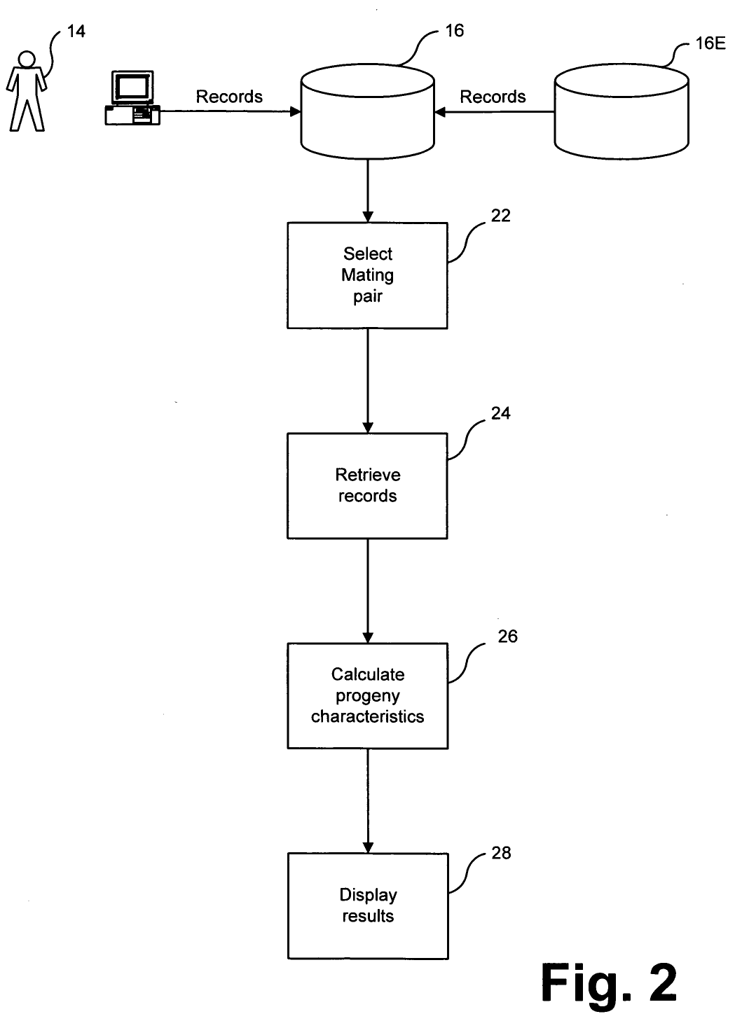

Charts 1 and 2 are attached hereto and incorporated herein by reference.

It is to be noted, however, that the appended drawings illustrate only typical embodiments of this invention and are therefore not to be considered limiting of its scope, for the invention may admit to other equally effective embodiments.

### DETAILED DESCRIPTION OF THE INVENTION

In the following description, numerous details are set forth to provide an understanding of the present invention. However, it will be understood by those skilled in the art that the present invention may be practiced without these details and that numerous variations or modifications from the described embodiments may be possible.

The present invention is herein described as a system and method of facilitating animal propagation. Referring to the Figures, the present invention is designed to utilize records pertaining to the offspring of a plurality of animals. In one embodiment, such records contain data relating to individual animals and their ancestors, relatives or offspring. Records may contain information relating to individual animal characteristics and/or traits. Characteristics and/or traits may encompass any animal quality worthy of consideration in making a mating determination, i.e., determining which male and female should be bred.

Records utilized by the present invention may be compiled and/or obtained in any reliable manner. In one embodiment, animal records compiled by reputable organizations, such as the American Angus Association, may be utilized. These records include EPD charts for individual animals and breed percentile EPD charts. Such information is utilized to assist animal owners and other interested parties in making an informed mating determination.

Upon selection of a potential or proposed mating pair, the present invention utilizes progeny predictors (11) associated with each animal to estimate the likelihood that the potential offspring of the mating pair will exhibit one or more desirable characteristics. In one embodiment, progeny predictors are predictions of the qualities of future offspring relative to the offspring of other animals.

In one embodiment, the calculation performed by the present invention involves taking the mathematical average of two actual progeny predictors (EPDs) of the proposed mating pair. The averaged progeny predictors of the mating pair provides an estimation of the extent the trait will be exhibited by the offspring of the proposed mating pair as compared to an average, should the user decide to breed these animals.

In one embodiment, the present invention may be embodied in one or more software applications capable of operating upon any number of hardware arrangements including, but not limited to, stand-alone devices and wireless devices. Such software applications may be loaded upon a computer system via any number of known methods, such as via CD-ROM or download via a computer network.

The present invention provides a processing unit (12) capable of accessing electronic records. Further, the processing unit has access to a plurality of web pages (12W) designed to assist remote users (14) in accessing the unique functionality of the present invention. In one embodiment, electronic records utilized by the processing unit of the present invention may be stored locally upon a storage device (16) and/or accessed through an external storage medium (16E). Access to the present invention may be provided via a computer network (18). Further, firewalls (20) may be utilized to control access to the processing unit and/or protect the system against harmful viruses.

In one embodiment, the present invention is accessible through an internet browser capable of connecting to the system via a computer network. Users are provided with a sign up procedure in which they provide identifying information and are assigned a username and password to ensure secure access, as illustrated by Box (17). Any number of internet browser applications may be utilized, including, but not limited to, those provided by Microsoft® and Netscape®.

Any number of known computer systems and/or electronic devices having internet access capability may load and display the web pages (12W) provided by the present invention. In one embodiment, the processing unit of the present invention contains a number of software packages designed to facilitate effective interaction with the user through his or her internet browser.

The present invention utilizes animal information or characteristics, including EPDs, from a number of sources, as illustrated by Box (36), thereby permitting the EPDs of a particular animal to be compared to industry standards or norms for the breed of animal. In one embodiment, a user (14) may provide or input into a computer system information or characteristics pertaining to specific animals in their possession, control, or ownership. Such information may be thought of as “internal” to a herd, ranch or organization since it pertains to specific animals of interest. Preferably, such information is quantified by a numeric value, thereby permitting comparison between animals and across groupings, herds, etc.

User (14) may also download or access “external” animal information or characteristics provided by external sources. As described above, a number of reputable organizations maintain information pertaining to particular types of animals. For example, the American Angus Association compiles animal information provided to them by ranchers around the country to generate progeny predictors commonly referred to as Expected Progeny Differences (EPDs). The graphic user interface (17) of the present invention provides a breed tab (19B) through which the user may select industry information for any number of animal breeds. In the examples provided herein involving the American Angus Association, the user would select “angus” upon the breed tab.

The progeny predictors utilized by the present invention vary depending on the type of animal at issue. Although the examples provided herein are directed to bovine animals, it should be understood that the functionality of the present invention may be applied to any number of types of animals. Further, the present invention may be utilized by breeders and/or animal owners utilizing any number of breeding techniques including, but not limited to, artificial insemination, natural service, and embryo transfer. In one embodiment, the present invention provides separate data entry registries for entry of animal information pertaining to animals subject to varying breeding techniques.

In one embodiment, the present invention utilizes one or more of the following EPDs: calving ease direct, birth weight, weaning weight, yearling weight, yearling height, scrotal circumference, calving ease maternal, maternal milk, mature weight, mature height, energy value, carcass weight, marbling, ribeye area, percent retail product, intramuscular fat, fat thickness, weaned value, feedlot value, grid value, and/or beef value. While these EPDs will be described in more detail below, those skilled in the art will understand that the invention is not limited to the foregoing EPDs and that the invention would work equally well for any quantifiable characteristic of an animal.

Calving Ease Direct (CED) is expressed as a difference in percentage of unassisted births, with a higher value indicating greater calving ease in first-calf heifers. CED predicts the average difference in ease with which an animal's calves will be born when bred to first-calf heifers. Birth Weight (BW), expressed in pounds, is a predictor of an animal's ability to transmit birth weight to his progeny compared to that of other animals. Weaning Weight (WW), expressed in pounds, is a predictor of an animal's ability to transmit weaning growth to his progeny compared to that of other animals. Yearling Weight (YW), expressed in pounds, is a predictor of an animal's ability to transmit yearling growth to his progeny compared to that of other animals.

Yearling Height (YH) is a predictor of an animal's ability to transmit yearling height, expressed in inches, compared to that of other animals. Scrotal Circumference (SC), expressed in centimeters, is a predictor of the difference in the animal's transmitting ability for scrotal size compared to that of other animals. Calving Ease Maternal (CEM) is expressed as a difference in percentage of unassisted births with a higher value indicating greater calving ease in first-calf daughters. It predicts the average ease with which an animal's daughters will calve as first-calf heifers when compared to daughters of other animals.

Maternal Milk (Milk) is a predictor of an animal's genetic merit for milk and mothering ability as expressed in its daughters as compared to daughters of other animals. In other words, it is that part of a calf's weaning weight attributed to milk and mothering ability. Mature Weight EPD (MW), expressed in pounds, is a predictor of the difference in mature weight of daughters of an animal compared to the daughters of other animals.

Mature Height (MH), expressed in inches, is a predictor of the difference in mature height of an animal's daughters compared to daughters of other animals. Energy Value ($EN), expressed in dollars savings per animal per year, assesses differences in animal energy requirements as an expected dollar savings difference in daughters of animals. A larger value is more favorable when comparing two animals (more dollars saved on feed energy expenses). Components for computing the $EN savings difference include lactation energy requirements and energy costs associated with differences in mature animal size.

Carcass Weight (CW), expressed in pounds is a predictor of the differences in hot carcass weight of an animal's progeny compared to progeny of other animals. Marbling (Marb), expressed as a fraction of the difference in USDA marbling score of an animal's progeny compared to progeny of other animals. Ribeye Area (RE), expressed in square inches, is a predictor of the difference in ribeye area of an animal's progeny compared to progeny of other animals. Fat Thickness (Fat), expressed in inches, is a predictor of the differences in external fat thickness at the 12th rib (as measured between the 12th and 13th ribs) of an animal's progeny compared to progeny of other animals.

Weaned Value ($W), is an index value expressed in dollars per head, is the expected average difference in future progeny performance for pre-weaning merit. $W includes both revenue and cost adjustments associated with differences in birth weight, weaning direct growth, maternal milk, and mature animal size.

Feedlot Value ($F), is an index value expressed in dollars per head, is the expected average difference in future progeny performance for post-weaning merit compared to progeny of other animals. Grid Value ($G), an index value expressed in dollars per head, is the expected average difference in future progeny performance for carcass grid merit compared to progeny of other animals.

Beef Value ($B), is an index value expressed in dollars per head, is the expected average difference in future progeny performance for post-weaning and carcass value compared to progeny of other animals. $Value indexes are multi-trait selection indexes, expressed in dollars per head, to assist beef producers by adding simplicity to genetic selection decisions. The $Value is an estimate of how future progeny of each animal are expected to perform, on average, compared to progeny of other animals in the database if the animal were randomly mated and if offspring calves were exposed to the same environment.

In one embodiment, the present invention may also utilize one or more of the following EPDs: total maternal, mature cow maintenance energy requirements, heifer pregnancy, calving ease total maternal, stayability, maternal milk & growth, maternal milk, baldy maternal index, Brahman influence index, certified Hereford beef index, calving ease index, yield grade, gestation length, all purpose index, terminal index, grid merit, feedlot merit, docility, and/or mainstream terminal index.

Total Maternal (TM) is measured in pounds of calf weaned by an animal's daughters. TM accounts for average differences that can be expected from both weaning weight direct as well as from milk, and measure an animal's ability to transmit milk production and growth rate through its daughters. TM is calculated by adding an animal's Milk predictor to one-half of its Weaning Weight. Mature Cow Maintenance Energy Requirements (ME) is an assessment of the differences in animal's energy requirements.

Heifer Pregnancy (HPG) is an estimate of the differences in an animal's daughters' ability to conceive to calve as a two year old. HPG is expressed in terms of a percentage difference. Calving Ease Total Maternal (CETM) is expressed as a difference in percentage of unassisted births with a higher value indicating greater calving ease in first-calf daughters. CETM predicts the average ease with which an animal's daughters will calve as first-calf heifers when compared to daughters of other animals.

Stayability (ST) is expressed as the probability that an animal's daughters will remain in production to at least six years of age when compared to the daughters of another animal. A measure of sustained fertility that probably reflects traits such as fleshing ability and structural soundness. Expressed as deviations from a 50% probability, a higher value indicates increased stayability.

Maternal Milk & Growth (MG) expresses what the animal is expected to transmit to its daughters for a combination of growth genetics through weaning and genetics for milking ability. MG is an estimate of daughters' progeny weaning weight. Maternal Milk (MM) expresses the milking ability of an animal's daughters expressed in pounds of calf weaned. It predicts the difference in average weaning weight of animal's daughters' progeny due to milking ability.

Baldy Maternal Index (BMI$) is an index designed to maximize profit for commercial calf producers that use Hereford bulls in rotational crossbreeding programs on Angus based cows and heifers. Retained ownership of calves through the feedlot phase of production is assumed, with fed cattle marketed on a Certified Hereford Beef (CHB) program pricing grid.

Brahman Influence Index (BII$) is similar to BMI$, with one primary difference. Hereford bulls are used in rotational crossing with Brahman, rather than Angus. This index places greater emphasis on traits deficient in Brahman cross cattle, such as fertility and age at puberty, and less on traits that are more acceptable in those cattle, such as growth and calving ease.

Certified Hereford Beef Index (CHB$) is a terminal animal index, where Hereford bulls are mated to British cross cows and all offspring are sold as fed cattle on a CHB pricing grid. This index emphasizes growth at all stages along with carcass values. No emphasis is put on fertility or milk because replacements are not kept.

Calving Ease Index (CEZ$) is similar to BMI$, except that Hereford bulls are mated only to yearling heifers. This index has increased emphasis on direct and maternal calving ease compared with the other indexes. These indexes allow a breeder to utilize a specific target to make animal selections. The indexes also allow a breeder to select for a balance of traits without putting thresholds on individual traits. Yield Grade (YG) is a USDA yield grade estimate of beef carcass cutability, which is defined as the combined yield of closely trimmed, boneless retail cuts from the round, loin, rib and chuck.

Gestation Length (GL) predicts average differences in gestation length. Expressed in days. Shorter gestation lengths are associated with less dystocia and longer post-partum intervals. All Purpose Index (API$) evaluates animals being used on the entire cowherd (bred to both Angus first-calf heifers and mature cows) with a portion of their daughters being retained for breeding and the steers and remaining heifers being put on feed and sold grade and yield.

Terminal Index (TI$) evaluates an animal's economic merit in situations where they are bred to mature Angus animals and all offspring are placed in the feedlot and sold grade and yield. Grid Merit (GM) is the expected average difference in future progeny performance for carcass grid merit compared to progeny of other animals. Feedlot Merit (FM) is the expected average difference in future progeny performance for post-weaning merit compared to progeny of other animals.

Docility (DOC) is a prediction of the percentage of an animal's offspring that are expected to score favorably (1 or 2) on a five-point scoring system when compared to the offspring of another animal. Expressed as a percentage with higher values being favorable. Mainstream Terminal Index (MTI$) ranks animals under the assumption that Limousine-sired calves are sold into the commodity beef market with premiums and discounts based on both quality and yield.

As described above, the present invention provides an attractive graphic user interface (17) through which users may enter animal information (19). In one embodiment, such information may be categorized and stored according to ranch (19R), herd (19H), or pasture (19P), as illustrated by Box (37). Once animal information, including individual animal EPDs, has been made available to the system, the user may browse a plurality of animal records and select a potential mating pair, as illustrated by Box (22). Once a potential mating pair has been selected, the processing unit of the present invention accesses animal information pertaining to each animal of the mating pair and performed the mating calculation, as illustrated by Box (26). Results of the mating calculation are then displayed for review by the user as illustrated by Box (28).

In one embodiment, the present invention provides a comprehensive animal propagation tool designed to provide the user with multiple mating options. In this embodiment, the storage device of the present invention is populated with industry information containing progeny predictors for a plurality of animals in addition to the information provided by the user regarding his or her herd. In one embodiment, the system accesses and stores industry animal information. Such information provides progeny predictors for hundreds or thousands of breed specimens and may be accessed by the system in any known manner.

This feature of the present invention allows the system to conduct mating calculations for hundreds of potential mating specimens. For example, if the user enters information relating to a single female specimen, he may then select a group of male specimens taken from a reliable external information source. With a simple click of the mouse button, the system is capable of running calculations using progeny predictors of the female specimen owned by the user and the progeny predictors of a plurality of male specimens stored upon the system or accessed from an external source.

In one embodiment, a single mating calculation is performed, then one of the animals, typically the bull, within the selected mating pair is replaced, as illustrated by Box (26R). This process may be repeated until all of the males in the group selected by the user have been calculated in comparison to the user's female animal. Comparative results (21) may then be displayed upon the graphic user interface (17) for review by the user.

Prior to running a mating calculation, the user may enter recall or restore offspring preference information, as illustrated by Box (39). For example, if the user's business objective is to have animal offspring having a high weaning weight, described further below, he or she may prioritize (or emphasize) (23) and/or (25) this offspring characteristic prior to running a mating calculation. In one embodiment, the user may prioritize one or more progeny predictors by assigning a percentage emphasis to one or more traits.

Further, the present invention allows the user within the given trait, emphasize maximum (27) or minimum (29) a particular characteristic or to optimize, as set forth in more detail below.

For example, it is often desirable to have animals exhibiting the lowest birth weight possible while, at the same time, having the highest weaning weight possible. In this example, the user would enter preference information maximizing the weaning weight predictor while minimizing the birth weight predictor prior to starting the mating calculation.

Animal industry information typically has an accuracy associated thereto. In short, the accuracy of industry information relating to a particular breed of animal may vary depending on the amount of information available regarding the animal's offspring and their individual performance data. Typically, the more data relating to a particular animal's offspring, the greater the accuracy of the progeny predictor associated with that animal.

In one embodiment, the user may assign a minimum accuracy value (30) to any and all of the progeny predictors utilized by the present invention in culling animals before conducting a mating calculation. For example, if the user only wishes to consider animals having a minimum accuracy value of greater than 75%, the user would enter “0.75” into the data field corresponding to the progeny predictor at issue.

In one embodiment, the user's offspring preferences are taken into account and utilized by the system during the mating calculation(s), to determine projected offspring EPDs as illustrated by Box (40). Each mating calculation to determine projected offspring EPDs may be assigned a score (31) based upon the most likely qualities of the offspring as illustrated by Box (26C). In one embodiment, a score of between 0 and 100 is assigned to each potential mating pair.

Mating scores may be ranked (32) and/or graphed (34) in order to provide the user with a convenient arrangement of mating information from which to make his or her mating decisions. Such information may be stored for later use, as illustrated by Box (41).

The mating calculation(s) made possible by the unique functionality of the present invention utilize an average of the progeny predictors for the male and female of a mating pair to generate projected offspring progeny predictor values. For example, a male animal having a weaning weight predictor having a value of 43 matched with a female animal having a weaning weight predictor having a value of 37 would generate a calculated offspring weaning weight predictor value of 40.

After calculating the offspring's predictor values, the present invention continues by calculates a mating score (31) for the proposed match. To accomplish this, the system first selects the predictors indicated as important by the user, either through prioritization, optimization, maximization, and/or minimization. For example, let's assume for the purposes of illustration that the user selects the following offspring preferences:

Birth weight=15% priority, minimized,

Weaning weight=30% priority, maximized

Milk=10% priority, optimized with an optimized value of 20

% IMF=15% priority, maximized, and

$W=30% priority, maximized.

Note that the % priority entered by the user must equal 100%. In one embodiment, only the predictors set forth by the user in this example, i.e., BW, WW, Milk, % IMF, and $W will be utilized in calculating a mating score.

Using the example set forth in paragraphs 60-66 above, the steps, tables, and charts below will help illustrate an embodiment of the novel method. The problem addressed is: a rancher has a cow, for example, cow “C” and wants to mate it with one bull from a set of available bulls, for example, bulls “W,” “X,” “Y,” and “Z.” Cow C and bulls W, X, Y and Z each have various characteristics that can be numerically quantified and used to make comparisons between the animals and pairings of the animals. These quantifiable characteristics are typically expressed as “EPDs” and measured with an EPD value or number. Thus, each animal can be said to have an associated set of EPDs covering a set of progeny characteristics. Applicant's method, in part, seeks to utilize these EPD values to score and rank each potential mating pair: C-W; C-X; C-Y; and C-Z, as to reflect the probability that a chosen weighed and prioritized criteria set of desired traits has been achieved. The steps may, typically, include the following:

Step 1: Choose a set of quantifiable characteristics or EPDs for consideration. Here, the EPD criteria set of 5 EPDs is chosen from a larger set of EPDs. Once a desired EPD criteria set is chosen, the actual EPD values for each animal are obtained and used to populate a matrix such as set forth in Table 1 below. As mentioned above, a rancher or other individual generally maintains this “internal” information for specific animals in his control, ownership or possession. In this example, the rancher has determined that the following EPD traits are important in the desired offspring: birth weight (BW), weaning weight (WW), maternal milk (M), weaned value ($W), and intramuscular fat (% IMF). Optionally, the animals may be culled for those falling below a certain accuracy, for example, 0.75. Here, the accuracy values of the EPDs of the four bulls are all 0.75 or above.

TABLE 1
Actual Animal EPD Values

| | C | W | X | Y | Z |
|---|---|---|---|---|---|
| EPD1 BW | 2 | −1.1 | 4.6 | 2 | 1.2 |
| EPD2 WW | 34 | 18 | 64 | 40 | 42 |
| EPD3 M | 28 | 15 | 24 | 25 | 11 |
| EPD4 IMF | 0.18 | 0.2 | −0.22 | 0.3 | 0.44 |
| EPD5 $W | 25.2 | 28.5 | 11.3 | 22.4 | 30.15 |

Step 2: Next, the various EPDs to be considered are weighted or “prioritized” based on their significance to the rancher in the particular evaluation, thereby emphasizing among the chosen EPDs, those EPDs considered to be the most significant. The rancher chooses, based on need, the relative emphasis desired for each of the EPD traits. From the example above (paragraphs 60-64), Step 2 discloses the following emphasis placement: BW 15%; WW 30%; milk 10%; IMF 15%; and $W 30%. The rancher here quantifies relative importance of each of the five traits (as compared to the others), the total always being 100%. Here, weaning weight and weaned value are equally emphasized (30%), both more important than birth weight (15%), intramuscular fat (15%), and milk (10%).

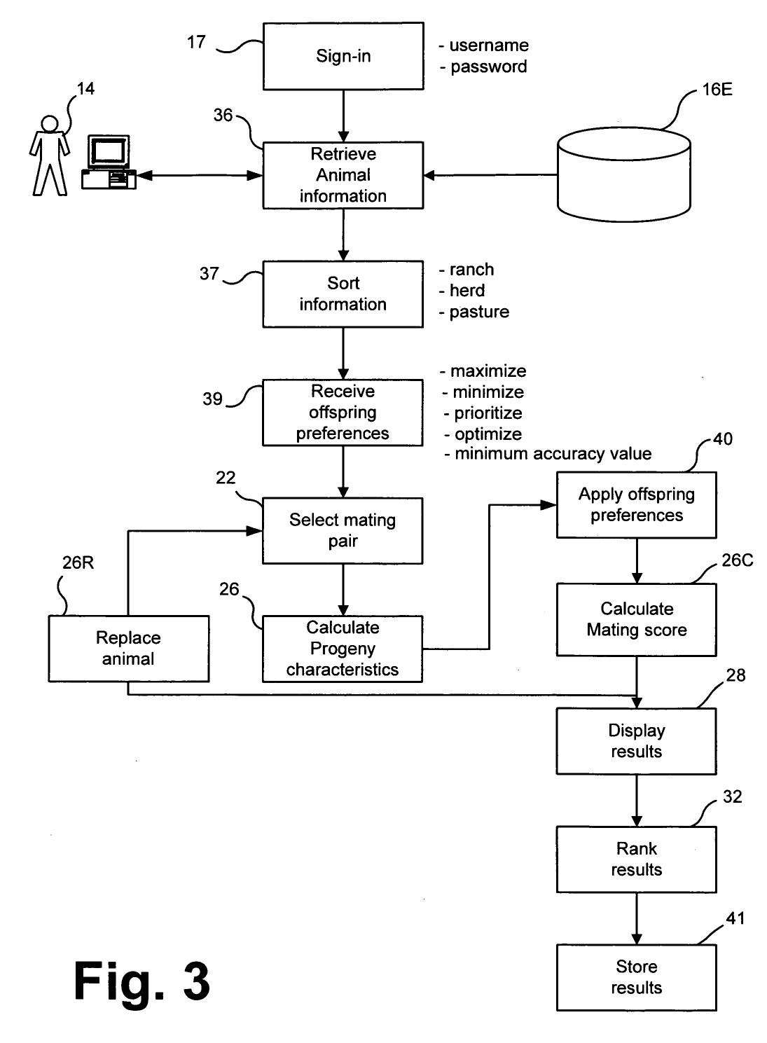

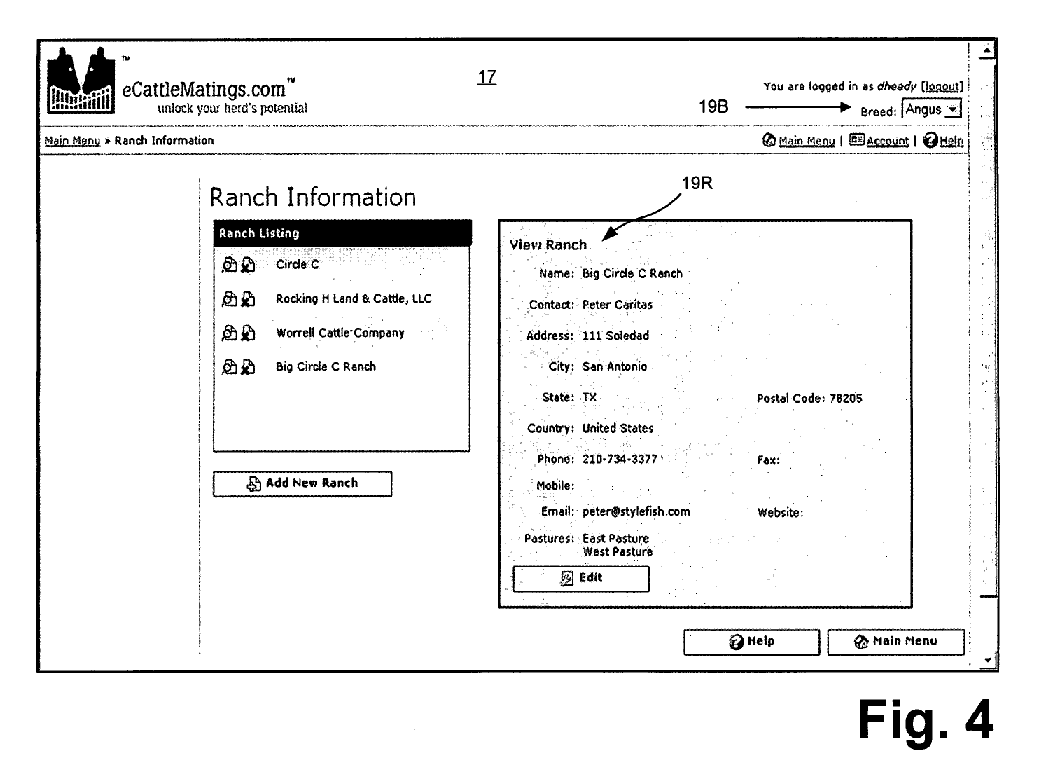

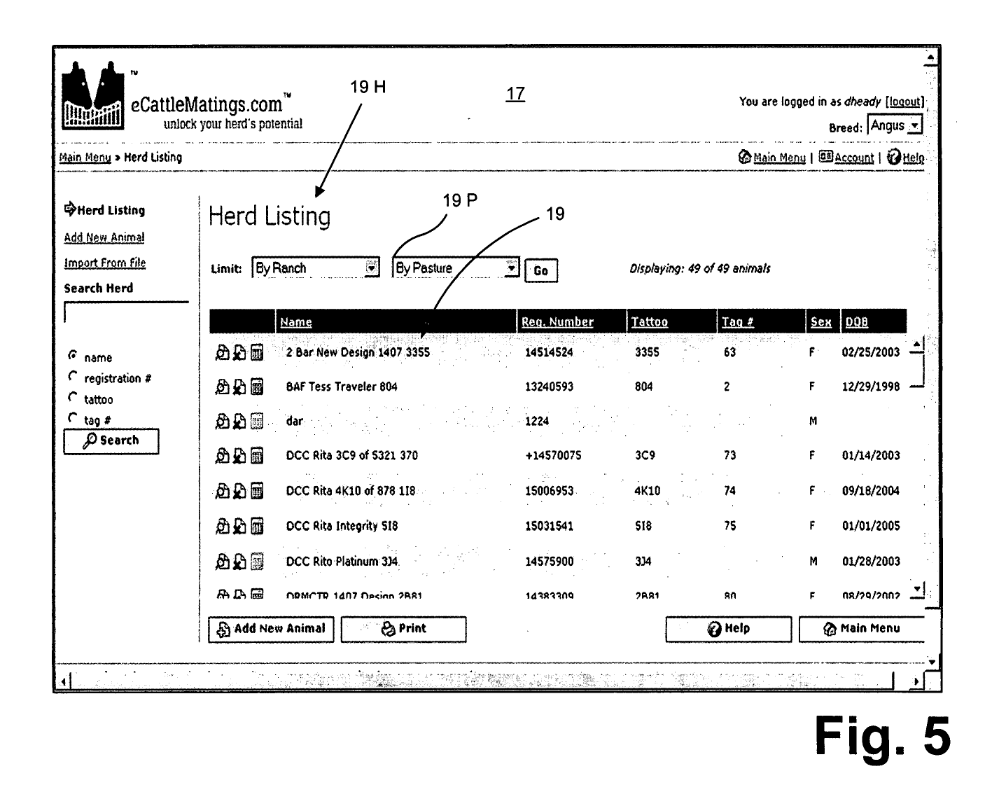

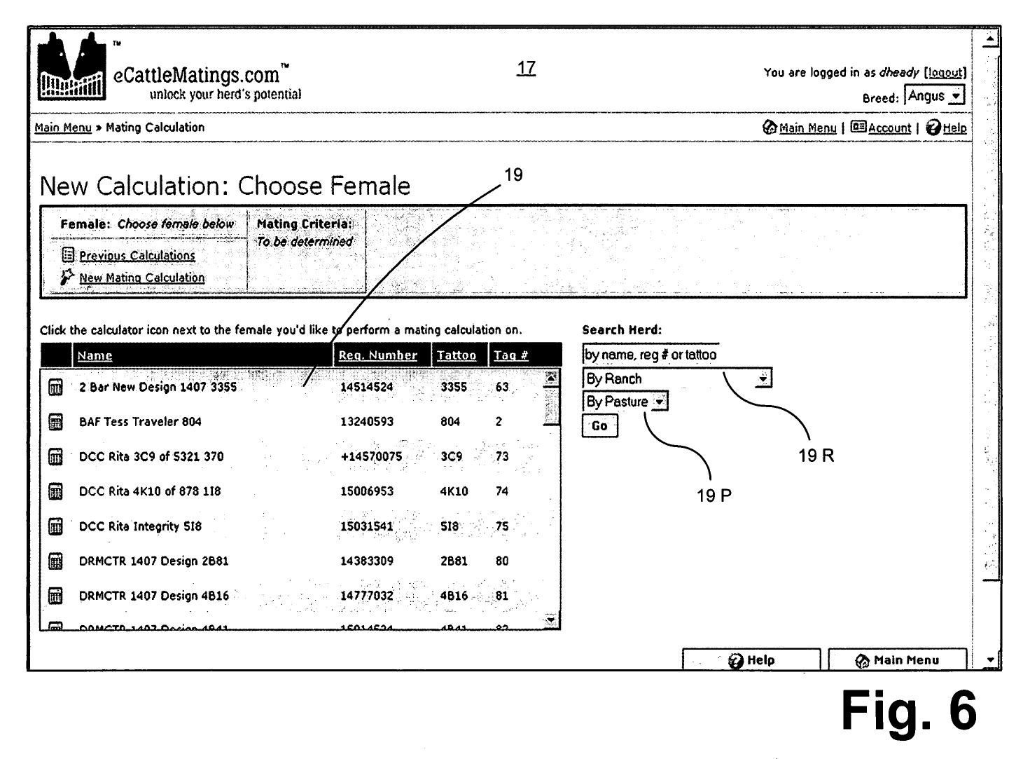

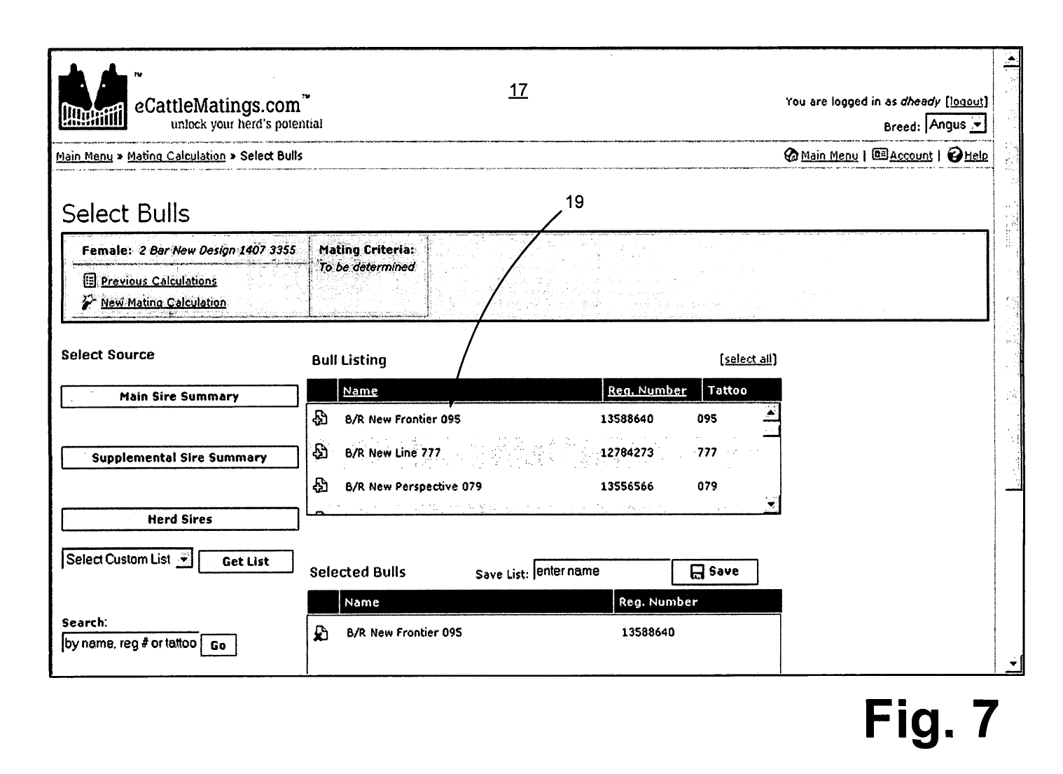

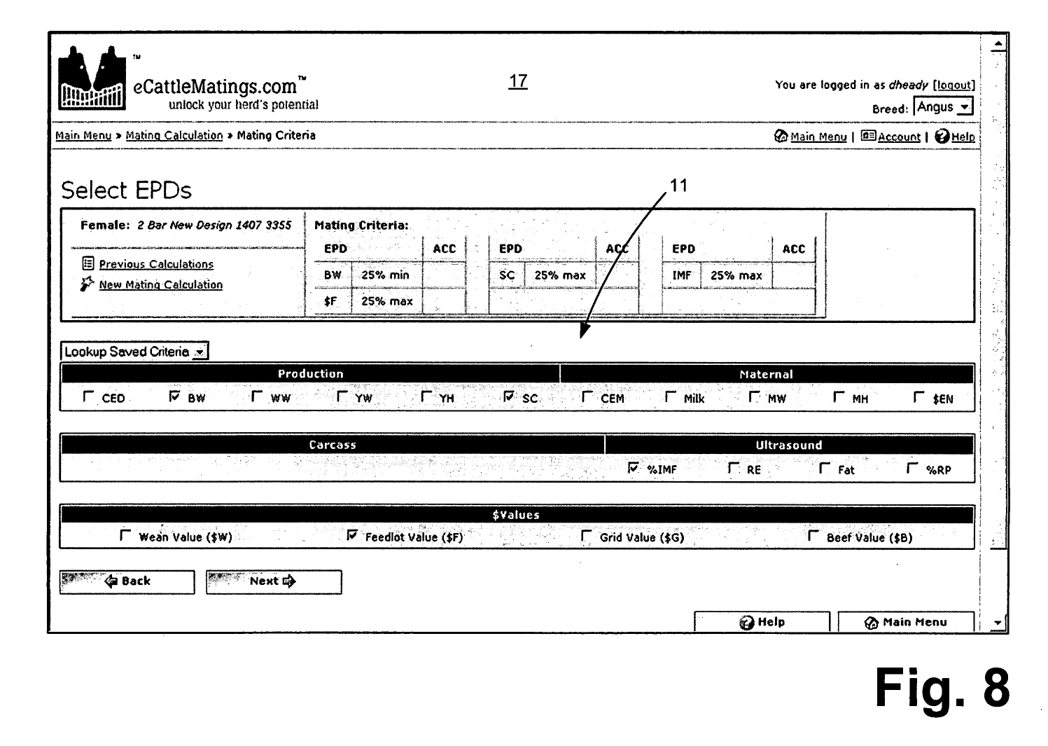

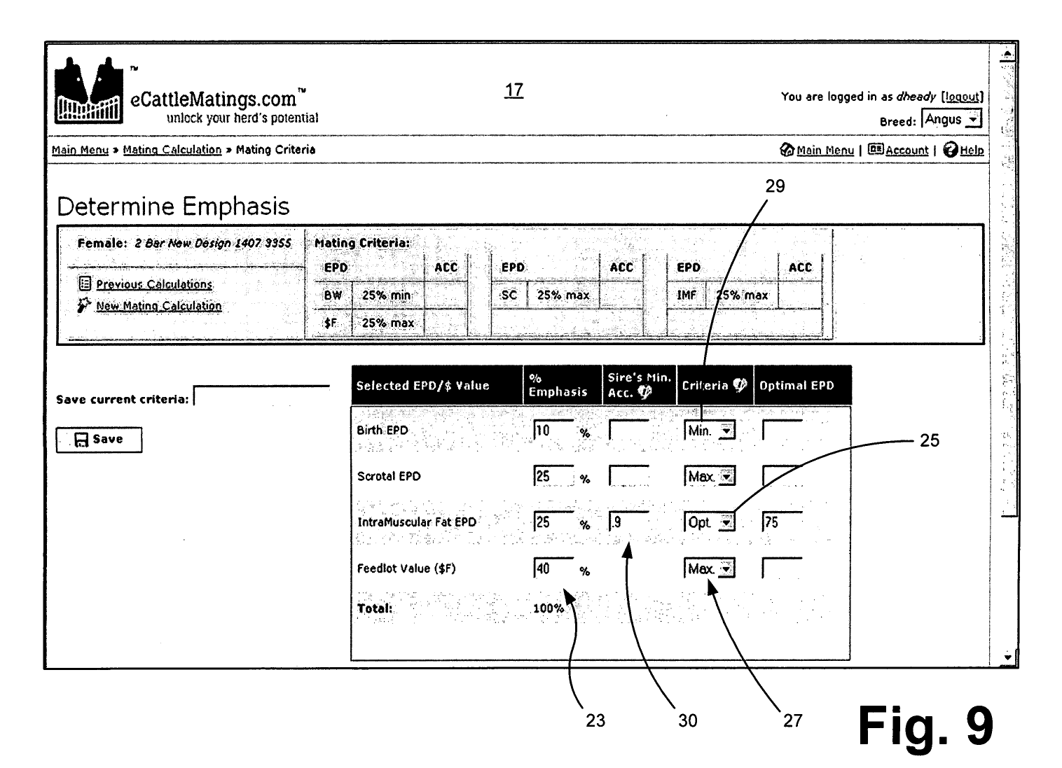

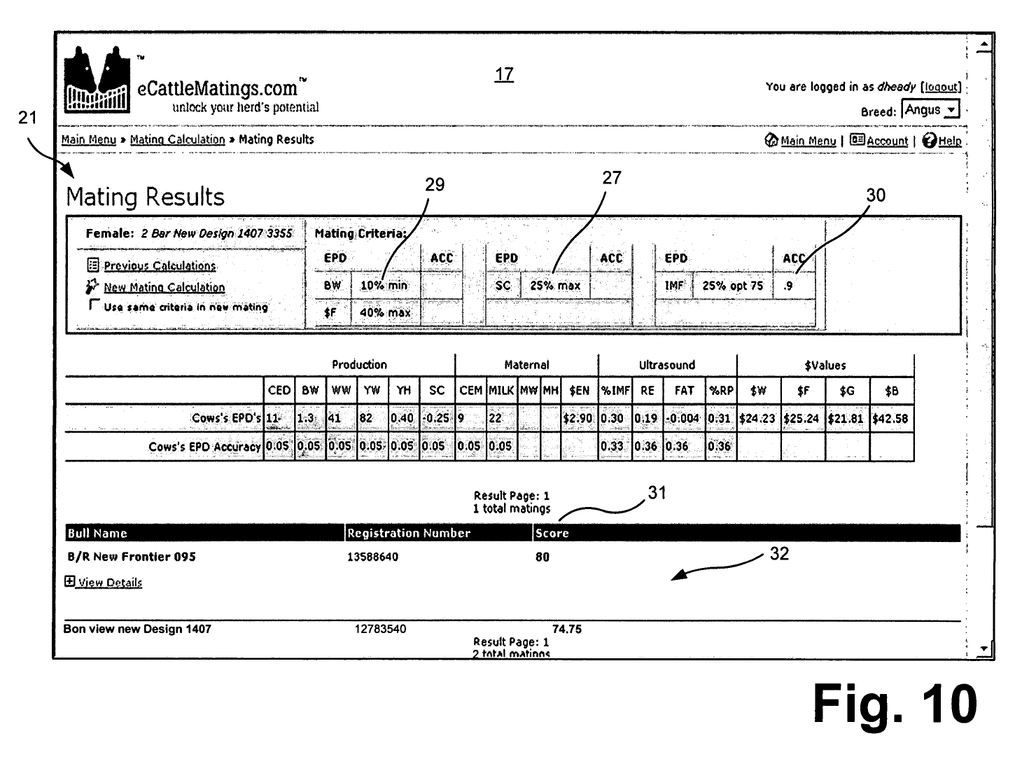

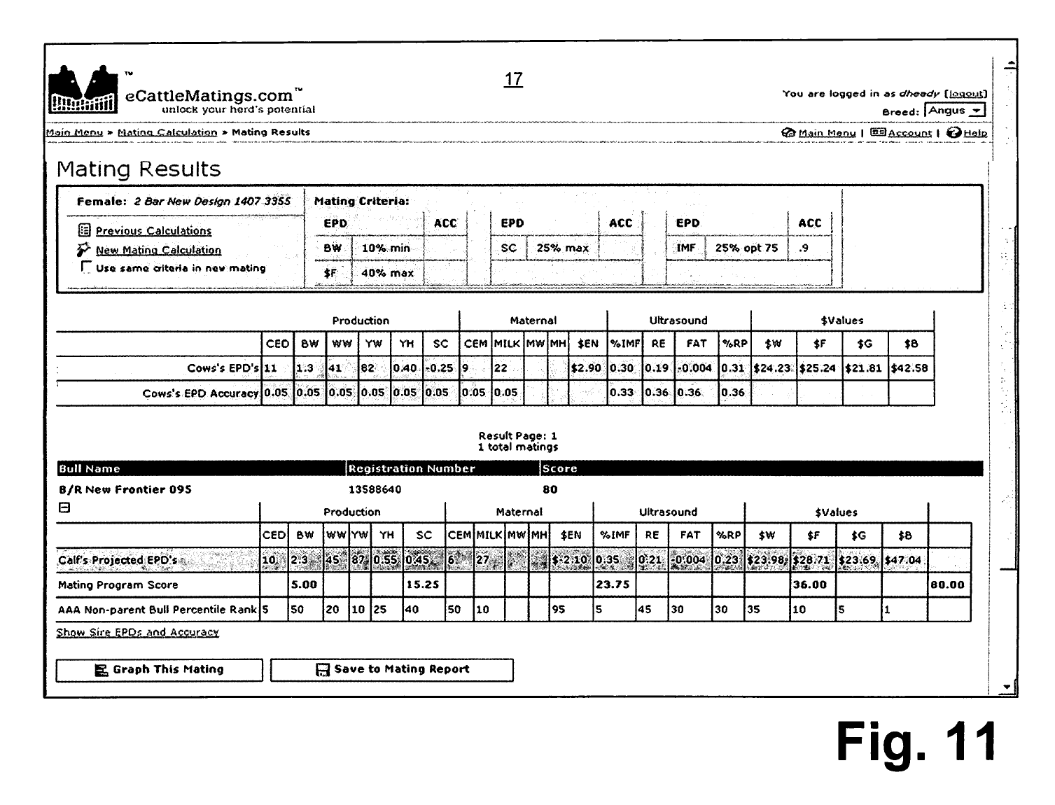

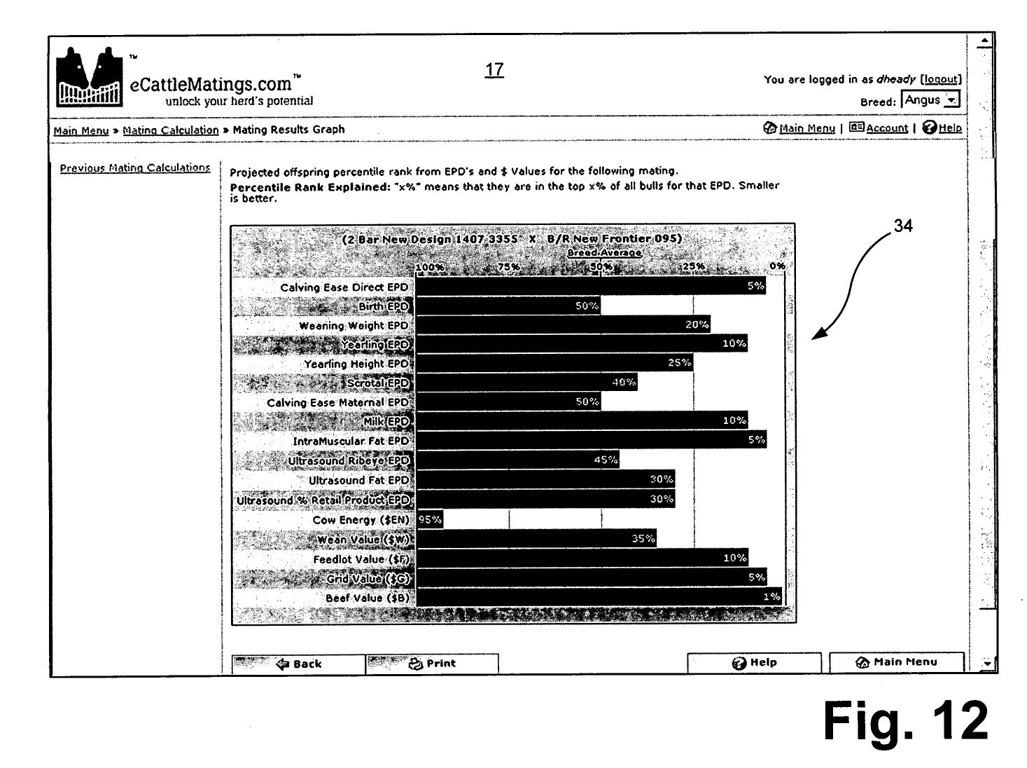

Step 3: At this step, the decision is made as whether to “maximize”, “minimize” or “optimize” each of the animals' quantifiable characteristics represented by EPDs. This step determines how an animal's percentile score (see Step 5 below) for a particular EPD will be derived. In the example, the rancher, based on perceived needs, chose to minimize BW (emphasizing low birth weight); maximize WW (emphasizing maximum weaning weight); optimize milk (with an optimum value of 20); maximize % IMF (emphasizing percent of intramuscular fat); and maximize $W (maximizing weaning value). When the rancher chooses to optimize he/she will also choose a selected optimum value, rather than minimize or maximize, based on perceived need. The optimum value, as described in more detail below is a preferred or desired mating calculation value for use in determining EPD percentile scores (as opposed to the “actual” mating calculation value which is derived below in Step 4). See FIG. 3, Box 39, for Steps 1, 2, and 3.

Step 4: This step determines a “projected offspring” EPD for each potential cow-bull pair. This projected offspring EPD is expressed numerically as a mating calculation value. More particularly, this step performs a mathematical calculation utilizing the actual EPD values from Table 1 for each potential pair (the group defining a set of potential pairs): C-W; C-X; C-Y; and C-Z. The result of the mathematical calculation is the mating calculation value. In one preferred embodiment, the mathematical calculation simply finds the average actual EPD value for each potential pair. In any event, the mating calculation value determines the projected offspring EPDs for each potential pair. This is done for each of the five selected traits, EPD1 thru EPD5, as set forth in the table below, for each of the four potential matings. In the above described preferred embodiment, the actual values for EPDs are added together and divided by two for each animal pairing. For example, EPD1 (BW) for the cow is 2.0 pounds, for bull W, it is −1.1. The sum is 0.90, which when divided by 2 equals 0.45 projected offspring EPD (see Box 26, FIG. 3). All EPDs for potential mating pairs may be calculated in this manner. What this has accomplished is quantified how the likelihood that potential offspring, as to each EPD trait, will compare to potential offspring for other pairings.

TABLE 2
Projected Offspring EPD

| | C-W | C-X | C-Y | C-Z |
|---|---|---|---|---|
| EPD1 BW | 0.45 | 3.30 | 2.00 | 1.60 |
| EPD2 WW | 26.00 | 49.00 | 37.00 | 38.00 |
| EPD3 M | 21.50 | 26.00 | 26.50 | 19.50 |
| EPD4 IMF | 0.19 | −0.02 | 0.24 | 0.31 |
| EPD5 $W | 26.85 | 18.25 | 23.80 | 27.68 |

Step 5: The next step assigns a “percentile score” to each projected offspring EPD for each pairing, preferably based on industry wide EPD numbers for a particular breed. The percentile scores may be derived mathematically utilizing various formulas capable of predicting industry averages or, alternatively, determined from a simple “lookup” table, such as illustrated by Charts 1 and 2. A percentile score can be looked up for each projected offspring EPD. The particular chart to reference for the percentile score of a given projected offspring EPD value may be selected based on whether that mating calculation was maximized, minimized or optimized in Step 3. Essentially, Step 5 converts the projected offspring EPD value into a percentile score standardized based on industry averages for a particular breed. For example, EPD1 BW was minimized, so we would go to the appropriate BW/minimize chart and for each of the mating calculations: 3.5; 3.0; 5.0; and 4.25; obtain the percentile conversion. Each chart is 0-100% based on percentile. Thus, Table 3 shows the mating calculations of Table 2, converted to percentages based on whether maximum, minimum or optimization is chosen. What this has accomplished is to compare the mating calculation to the breed percentiles (typically the available non-father male EPD percentile chart is used), allowing a comparison of where, breed percentile-wise, the projected offspring EPD BW (or other trait) for C-W stands (in the 86^{th} percentile) as to the other three potential pairs, for example, (here, C-X is lowest at 25%). The results obtained may be termed a trait mating score.

TABLE 3
Conversion to Trait Mating Scores

| | C-W | C-X | C-Y | C-Z |
|---|---|---|---|---|
| EPD1 BW | 86 | 25 | 57 | 67 |
| EPD2 WW | 6 | 88 | 36 | 41 |
| EPD3 M | 87 | 60 | 58 | 97 |
| EPD4 IMF | 71 | 21 | 79 | 88 |
| EPD5 $W | 80 | 11 | 51 | 86 |

The lookup of Step 5 may be accomplished utilizing the attached Chart 1 (minimize/optimize) or Chart 2 (maximize). These charts are an expanded form of charts available for particular breeds, Charts 1 and 2 expanded and adapted from the percentile EPD breed chart for the Angus breed available online at the American Angus Association website and updated, typically every six months. Applicants' charts are adapted from the American Angus Association charts and thus this example is specific to that breed. The American Angus Association charts are provided for non-parent bulls and cows and parent bulls and cows. Since the sex of the projected offspring is not known and since it is not born a mother or father, Applicants preferably adopt the non-parent bulls EPD percentile chart. Further, the charts attached are expanded from the current American Angus Association percentile charts, in that one does not have to interpolate as much, for example, where an actual or calculated EPD might fall on the available charts between 90-95%, Applicants' expanded form interpolates between 90-95%, making it easier to get accuracy.

To optimize a mating calculation value and hence a percentile score, go to the appropriate EPD column (for example, BW) and locate the percentile rank of the calculated mating calculation value for that EPD trait. Next, it is determined how many percentage points the calculated or actual mating calculation value is away from the chosen optimum mating value, and subtract that difference from 100, to obtain the result in Step 6. In the present example, the only trait chosen for optimization is milk, which was optimized at 20. Going to Chart 1, for mating pair C-W, a projected offspring EPD calculation for milk was 21.50. On Chart 1, the optimized value of 20 for milk corresponds to the 50th percentile. The mating calculation for milk, for the C-W potential pairing is 21.50, which corresponds to about the 37 percentile on Chart 1. The difference between 50 percentile and 37 percentile is 13. Subtracting 13 percentile from 100 percentile yields the 87, which is the conversion value or percentile score shown in Table 3, column C-W, row 3, for milk.

Step 6: Once trait mating scores are determined (see Box 40, FIG. 3), the priority percentages selected in Step 2 are applied to yield a “weighted value” or prioritized trait mating score (see Box 26C, FIG. 3) for each percentile score. Specifically, the percentile score value from Step 5 is multiplied by the percentage priority chosen in Step 2 to reach a weighed value for each converted mating calculation of Table 3. These weighed values are set forth in Table 4. For example, CW BW had a mating calculation that converted into 86%. The priority weight given BW is 15%. 85×0.15=12.9 as noted in Table 4. This allows the user to apply the relative priority among the five EPDs.

TABLE 4
Prioritized Trait Scores

| | C-W | C-X | C-Y | C-Z |
|---|---|---|---|---|
| EPD1 BW | 12.9 | 3.75 | 8.55 | 10.05 |
| EPD2 WW | 1.8 | 26.4 | 10.8 | 12.3 |
| EPD3 M | 8.7 | 6 | 5.8 | 9.7 |
| EPD4 IMF | 10.65 | 3.15 | 11.85 | 13.2 |
| EPD5 $W | 24 | 3.3 | 15.3 | 25.8 |
| Summed: | 58.05 | 42.6 | 52.3 | 71.05 |

Step 7: The next step, which is also illustrated in Table 4, is to sum the prioritized trait mating scores for all five traits to get a combined trait mating score (or “mating score”) (see Box 26C, FIG. 3) for each cow-bull combination or pairing, and to rank all of the pairings from highest to lowest (see Box 32, FIG. 3). The highest combination graded scores would prove the most beneficial mating, achieving the greatest likelihood that this pair will produce offspring with the selected, prioritized traits. Summing results in a “composite view” of offspring potential tailored to the rancher's need (see Box 32, FIG. 3). Here, the rancher wanted offspring with low BW, high WW, IMF and $W and an optimum (20) for milk. Let's look at how they scored.

The best overall mating score for the selected, prioritized criteria is cow C with bull Z, the worst is cow C with bull X. While the rancher wanted offspring with low birth weight, Z did not have the lowest EPD for BW, that was W. However, birth weight was not the only trait in issue. High weaning weight was important. While Z only had the second lowest birth rate (W was lowest), the weaning weight for Z was much higher at 42 for bull Z than for bull W at 18. Bull Z also pulled ahead of bull W as a result of the emphasis (30%) of weaning weight as compared to birth weight. Of course, if the rancher decided to emphasize birth weight at much higher than 15% and weaning weight much lower, it could have changed the scoring. Likewise, if he wanted to minimize weaning weight rather than maximize it, that also could have likely changed the scoring.

Further, all of the steps above and as described herein can be provided through inputting into the computer through the user interface the selected animals, which each have the available EPDs. The selected EPD criteria set would also be input, as would the prioritization, and maximization, minimization or optimization.

The steps illustrated above are only exemplary, nor is it necessary to be performed in exactly the order set forth. For example, a rancher could first run the calculations for projected offspring EPD, then decide to maximize, minimize or optimize, perform this step and then decide how to prioritize. Further, the examples set forth above has illustrated only the novel method in comparing a single cow to four potential bulls for mating purposes. However, the female may, for example, be a composite of a herd of females. Likewise, the computerized method herein may perform a search for bulls from a predetermined herd or group, using the method set forth herein. While the Angus breed is used, it is only exemplary. EPD values for animals in other herds and herd chart averages and percentile charts are also available for other breeds or animal families or species.

Although the invention has been described with reference to specific embodiments, this description is not meant to be construed in a limited sense. Various modifications of the disclosed embodiments, as well as alternative embodiments of the invention, will become apparent to persons skilled in the art upon reference to the description of the invention. It is, therefore, contemplated that the appended claims will cover such modifications that fall within the scope of the invention.

CHART 1
Minimum/Optimum Chart

| Points | CED | BW | WW | YW | YH | SC | CEM | Milk | $EN | IMF | RE |
|---|---|---|---|---|---|---|---|---|---|---|---|
| 1 | 13 | 12.1 | 58 | 105 | 1.1 | 1.57 | 12 | 31 | 21.65 | 0.57 | 0.77 |
| 2 | 12 | 10.66 | 56 | 101 | 1 | 1.41 | 11 | 30 | 19.51 | 0.51 | 0.7 |
| 3 | 11 | 9.22 | 55 | 99 | 0.9 | 1.31 | 11 | 29 | 18.24 | 0.47 | 0.65 |
| 4 | 11 | 7.78 | 54 | 97 | 0.9 | 1.23 | 11 | 29 | 17.32 | 0.44 | 0.62 |
| 5 | 11 | 4.9 | 53 | 96 | 0.8 | 1.17 | 10 | 28 | 16.55 | 0.42 | 0.6 |
| 6 | 10.6 | 4.78 | 52.4 | 95 | 0.78 | 1.132 | 10 | 27.6 | 16.072 | 0.404 | 0.582 |
| 7 | 10.2 | 4.66 | 51.8 | 94 | 0.76 | 1.094 | 10 | 27.2 | 15.594 | 0.388 | 0.564 |
| 8 | 9.8 | 4.54 | 51.2 | 93 | 0.74 | 1.056 | 10 | 26.8 | 15.116 | 0.372 | 0.546 |
| 9 | 9.4 | 4.42 | 50.6 | 92 | 0.72 | 1.018 | 10 | 26.4 | 14.638 | 0.356 | 0.528 |
| 10 | 9 | 4.3 | 50 | 91 | 0.7 | 0.98 | 10 | 26 | 14.16 | 0.34 | 0.51 |
| 11 | 9 | 4.22 | 49.6 | 90.4 | 0.7 | 0.956 | 9.8 | 25.8 | 13.824 | 0.33 | 0.498 |
| 12 | 9 | 4.14 | 49.2 | 89.8 | 0.7 | 0.932 | 9.6 | 25.6 | 13.488 | 0.32 | 0.486 |
| 13 | 9 | 4.06 | 48.8 | 89.2 | 0.7 | 0.908 | 9.4 | 25.4 | 13.152 | 0.31 | 0.474 |
| 14 | 9 | 3.98 | 48.4 | 88.6 | 0.7 | 0.884 | 9.2 | 25.2 | 12.816 | 0.3 | 0.462 |
| 15 | 9 | 3.9 | 48 | 88 | 0.7 | 0.86 | 9 | 25 | 12.48 | 0.29 | 0.45 |
| 16 | 8.8 | 3.84 | 47.8 | 87.6 | 0.68 | 0.84 | 9 | 24.8 | 12.23 | 0.282 | 0.44 |
| 17 | 8.6 | 3.78 | 47.6 | 87.2 | 0.66 | 0.82 | 9 | 24.6 | 11.98 | 0.274 | 0.43 |
| 18 | 8.4 | 3.72 | 47.4 | 86.8 | 0.64 | 0.8 | 9 | 24.4 | 11.73 | 0.266 | 0.42 |
| 19 | 8.2 | 3.66 | 47.2 | 86.4 | 0.62 | 0.78 | 9 | 24.2 | 11.48 | 0.258 | 0.41 |
| 20 | 8 | 3.6 | 47 | 86 | 0.6 | 0.76 | 9 | 24 | 11.23 | 0.25 | 0.4 |
| 21 | 7.8 | 3.54 | 46.8 | 85.6 | 0.6 | 0.742 | 8.8 | 23.81 | 1.016 | 0.244 | 0.392 |
| 22 | 7.6 | 3.48 | 46.6 | 85.2 | 0.6 | 0.724 | 8.6 | 23.61 | 0.802 | 0.238 | 0.384 |
| 23 | 7.4 | 3.42 | 46.4 | 84.8 | 0.6 | 0.706 | 8.4 | 23.41 | 0.588 | 0.232 | 0.376 |
| 24 | 7.2 | 3.36 | 46.2 | 84.4 | 0.6 | 0.688 | 8.2 | 23.21 | 0.374 | 0.226 | 0.368 |
| 25 | 7 | 3.3 | 46 | 84 | 0.6 | 0.67 | 8 | 2310.16 | 0.22 | 0.36 | |
| 26 | 7 | 3.26 | 45.6 | 83.6 | 0.58 | 0.656 | 8 | 23 | 9.97 | 0.214 | 0.354 |
| 27 | 7 | 3.22 | 45.2 | 83.2 | 0.56 | 0.642 | 8 | 23 | 9.78 | 0.208 | 0.348 |
| 28 | 7 | 3.18 | 44.8 | 82.8 | 0.54 | 0.628 | 8 | 23 | 9.59 | 0.202 | 0.342 |
| 29 | 7 | 3.14 | 44.4 | 82.4 | 0.52 | 0.614 | 8 | 23 | 9.4 | 0.196 | 0.336 |
| 30 | 7 | 3.1 | 44 | 82 | 0.5 | 0.6 | 8 | 23 | 9.21 | 0.19 | 0.33 |
| 31 | 6.8 | 3.06 | 43.8 | 81.6 | 0.5 | 0.584 | 7.8 | 22.8 | 9.016 | 0.184 | 0.322 |
| 32 | 6.6 | 3.02 | 43.6 | 81.2 | 0.5 | 0.568 | 7.6 | 22.6 | 8.822 | 0.178 | 0.314 |
| 33 | 6.4 | 2.98 | 43.4 | 80.8 | 0.5 | 0.552 | 7.4 | 22.4 | 8.628 | 0.172 | 0.306 |
| 34 | 6.2 | 2.94 | 43.2 | 80.4 | 0.5 | 0.536 | 7.2 | 22.2 | 8.434 | 0.166 | 0.298 |
| 35 | 6 | 2.9 | 43 | 80 | 0.5 | 0.52 | 7 | 22 | 8.24 | 0.16 | 0.29 |
| 36 | 6 | 2.86 | 42.8 | 79.6 | 0.48 | 0.508 | 7 | 21.8 | 8.084 | 0.156 | 0.284 |
| 37 | 6 | 2.82 | 42.6 | 79.2 | 0.46 | 0.496 | 7 | 21.6 | 7.928 | 0.152 | 0.278 |
| 38 | 6 | 2.78 | 42.4 | 78.8 | 0.44 | 0.484 | 7 | 21.4 | 7.772 | 0.148 | 0.272 |
| 39 | 6 | 2.74 | 42.2 | 78.4 | 0.42 | 0.472 | 7 | 21.2 | 7.616 | 0.144 | 0.266 |
| 40 | 6 | 2.7 | 42 | 78 | 0.4 | 0.46 | 7 | 21 | 7.46 | 0.14 | 0.26 |
| 41 | 5.8 | 2.66 | 41.8 | 77.8 | 0.4 | 0.446 | 7 | 21 | 7.304 | 0.136 | 0.254 |
| 42 | 5.6 | 2.62 | 41.6 | 77.6 | 0.4 | 0.432 | 7 | 21 | 7.148 | 0.132 | 0.248 |
| 43 | 5.4 | 2.58 | 41.4 | 77.4 | 0.4 | 0.418 | 7 | 21 | 6.992 | 0.128 | 0.242 |
| 44 | 5.2 | 2.54 | 41.2 | 77.2 | 0.4 | 0.404 | 7 | 21 | 6.836 | 0.124 | 0.236 |
| 45 | 5 | 2.5 | 41 | 77 | 0.4 | 0.39 | 7 | 21 | 6.68 | 0.12 | 0.23 |
| 46 | 5 | 2.46 | 40.8 | 76.6 | 0.4 | 0.378 | 6.82 | 0.8 | 6.522 | 0.116 | 0.224 |
| 47 | 5 | 2.42 | 40.6 | 76.2 | 0.4 | 0.366 | 6.62 | 0.6 | 6.364 | 0.112 | 0.218 |
| 48 | 5 | 2.38 | 40.4 | 75.8 | 0.4 | 0.354 | 6.42 | 0.4 | 6.206 | 0.108 | 0.212 |
| 49 | 5 | 2.34 | 40.2 | 75.4 | 0.4 | 0.342 | 6.22 | 0.2 | 6.048 | 0.104 | 0.206 |
| 50 | 5 | 2.3 | 40 | 75 | 0.4 | 0.33 | 6 | 20 | 5.89 | 0.1 | 0.2 |
| 51 | 4.8 | 2.26 | 39.8 | 74.6 | 0.38 | 0.318 | 6 | 19.8 | 5.738 | 0.096 | 0.196 |
| 52 | 4.6 | 2.22 | 39.6 | 74.2 | 0.36 | 0.306 | 6 | 19.6 | 5.586 | 0.092 | 0.192 |
| 53 | 4.4 | 2.18 | 39.4 | 73.8 | 0.34 | 0.294 | 6 | 19.4 | 5.434 | 0.088 | 0.188 |
| 54 | 4.2 | 2.14 | 39.2 | 73.4 | 0.32 | 0.282 | 6 | 19.2 | 5.282 | 0.084 | 0.184 |
| 55 | 4 | 2.1 | 39 | 73 | 0.3 | 0.27 | 6 | 19 | 5.13 | 0.08 | 0.18 |
| 56 | 4 | 2.06 | 38.8 | 72.8 | 0.3 | 0.258 | 6 | 19 | 4.972 | 0.076 | 0.174 |
| 57 | 4 | 2.02 | 38.6 | 72.6 | 0.3 | 0.246 | 6 | 19 | 4.814 | 0.072 | 0.168 |
| 58 | 4 | 1.98 | 38.4 | 72.4 | 0.3 | 0.234 | 6 | 19 | 4.656 | 0.068 | 0.162 |
| 59 | 4 | 1.94 | 38.2 | 72.2 | 0.3 | 0.222 | 6 | 19 | 4.498 | 0.064 | 0.156 |
| 60 | 4 | 1.9 | 38 | 72 | 0.3 | 0.21 | 6 | 19 | 4.34 | 0.06 | 0.15 |
| 61 | 3.8 | 1.86 | 37.8 | 71.6 | 0.3 | 0.196 | 5.8 | 18.8 | 4.184 | 0.058 | 0.144 |
| 62 | 3.6 | 1.82 | 37.6 | 71.2 | 0.3 | 0.182 | 5.6 | 18.6 | 4.028 | 0.056 | 0.138 |
| 63 | 3.4 | 1.78 | 37.4 | 70.8 | 0.3 | 0.168 | 5.4 | 18.4 | 3.872 | 0.054 | 0.132 |
| 64 | 3.2 | 1.74 | 37.2 | 70.4 | 0.3 | 0.154 | 5.2 | 18.2 | 3.716 | 0.052 | 0.126 |
| 65 | 3 | 1.7 | 37 | 70 | 0.3 | 0.14 | 5 | 18 | 3.56 | 0.05 | 0.12 |
| 66 | 3 | 1.66 | 36.8 | 69.6 | 0.28 | 0.126 | 5 | 17.8 | 3.39 | 0.046 | 0.114 |
| 67 | 3 | 1.62 | 36.6 | 69.2 | 0.26 | 0.112 | 5 | 17.6 | 3.22 | 0.042 | 0.108 |
| 68 | 3 | 1.58 | 36.4 | 68.8 | 0.24 | 0.098 | 5 | 17.4 | 3.05 | 0.038 | 0.102 |
| 69 | 3 | 1.54 | 36.2 | 68.4 | 0.22 | 0.084 | 5 | 17.2 | 2.88 | 0.034 | 0.096 |
| 70 | 3 | 1.5 | 36 | 68 | 0.2 | 0.07 | 5 | 17 | 2.71 | 0.03 | 0.09 |
| 71 | 2.8 | 1.44 | 35.8 | 67.6 | 0.2 | 0.056 | 5 | 16.8 | 2.54 | 0.026 | 0.082 |
| 72 | 2.6 | 1.38 | 35.6 | 67.2 | 0.2 | 0.042 | 5 | 16.6 | 2.37 | 0.022 | 0.074 |
| 73 | 2.4 | 1.32 | 35.4 | 66.8 | 0.2 | 0.028 | 5 | 16.4 | 2.2 | 0.018 | 0.066 |
| 74 | 2.2 | 1.26 | 35.2 | 66.4 | 0.2 | 0.014 | 5 | 16.2 | 2.03 | 0.014 | 0.057 |
| 75 | 2 | 1.2 | 35 | 66 | 0.2 | 0 | 5 | 16 | 1.86 | 0.01 | 0.05 |
| 76 | 1.8 | 1.14 | 34.6 | 65.4 | 0.18 | −0.016 | 4.8 | 16 | 1.654 | 0.004 | 0.044 |
| 77 | 1.6 | 1.08 | 34.2 | 64.8 | 0.16 | −0.032 | 4.6 | 16 | 1.447 | −0.002 | 0.038 |
| 78 | 1.4 | 1.02 | 33.8 | 64.2 | 0.14 | −0.048 | 4.4 | 16 | 1.242 | −0.008 | 0.032 |
| 79 | 1.2 | 0.96 | 33.4 | 63.6 | 0.12 | −0.064 | 4.2 | 16 | 1.036 | −0.014 | 0.026 |
| 80 | 1 | 0.9 | 33 | 63 | 0.1 | −0.08 | 4 | 16 | 0.83 | −0.02 | 0.02 |
| 81 | 0.8 | 0.84 | 32.8 | 62.4 | 0.1 | −0.1 | 4 | 15.8 | 0.618 | −0.024 | 0.01 |
| 82 | 0.6 | 0.78 | 32.6 | 61.8 | 0.1 | −0.12 | 4 | 15.6 | 0.406 | −0.028 | 0 |
| 83 | 0.4 | 0.72 | 32.4 | 61.2 | 0.1 | −0.14 | 4 | 15.4 | 0.194 | −0.032 | −0.01 |
| 84 | 0.2 | 0.66 | 32.2 | 60.6 | 0.1 | −0.16 | 4 | 15.2 | −0.017 | −0.036 | −0.02 |
| 85 | 0 | 0.6 | 32 | 60 | 0.1 | −0.18 | 4 | 15 | −0.23 | −0.04 | −0.03 |
| 86 | −0.2 | 0.52 | 31.6 | 59.4 | 0.08 | −0.204 | 3.8 | 14.6 | −0.506 | −0.046 | −0.04 |
| 87 | −0.4 | 0.44 | 31.2 | 58.8 | 0.06 | −0.228 | 3.6 | 14.2 | −0.782 | −0.052 | −0.05 |
| 88 | −0.60 | 36 | 30.8 | 58.2 | 0.04 | −0.252 | 3.4 | 13.8 | −1.057 | −0.058 | −0.06 |
| 89 | −0.8 | 0.28 | 30.4 | 57.6 | 0.02 | −0.276 | 3.2 | 13.4 | −1.334 | −0.064 | −0.07 |
| 90 | −1 | 0.2 | 30 | 57 | 0 | −0.3 | 3 | 13 | −1.61 | −0.07 | −0.07 |
| 91 | −1.4 | 0.06 | 29.2 | 55.8 | −0.02 | −0.336 | 2.6 | 12.6 | −2.014 | −0.07 | −0.098 |
| 92 | −1.7 | −0.08 | 28.4 | 54.6 | −0.04 | −0.372 | 2.2 | 12.2 | −2.417 | −0.09 | −0.116 |
| 93 | −2.2 | −0.22 | 27.6 | 53.4 | −0.06 | −0.408 | 1.8 | 11.8 | −2.822 | −0.1 | −0.134 |
| 94 | −2.6 | −0.36 | 26.8 | 52.2 | −0.07 | −0.444 | 1.4 | 11.4 | −3.226 | −0.11 | −0.152 |
| 95 | −3 | −0.5 | 26 | 51 | −0.1 | −0.48 | 1 | 11 | −3.63 | −0.12 | −0.17 |
| 96 | −6.2 | −0.7 | 20 | 39.6 | −0.32 | −0.892 | −1.6 | 7.4 | −6.336 | −0.178 | −0.33 |
| 97 | −9.4 | −0.9 | 14 | 28.2 | −0.54 | −1.304 | −4.2 | 3.8 | −9.042 | −0.236 | −0.49 |
| 98 | −12.6 | −1.2 | 8 | 16.8 | −0.76 | −1.716 | −6.8 | 0.2 | −11.747 | −0.294 | −0.65 |
| 99 | −15.8 | −1.8 | 2 | 5.4 | −0.97 | −2.128 | −9.4 | −3.4 | −14.454 | −0.352 | −0.81 |
| 100 | −19 | −99 | −4 | −6 | −1.2 | −2.54 | −12 | −7 | −17.16 | −0.41 | −0.97 |

| Points | Fat | % RP | $W | $F | $G | $B |
|---|---|---|---|---|---|---|
| 1 | 0.099 | 0.78 | 32.06 | 43.51 | 28.74 | 50.77 |
| 2 | 0.085 | 0.69 | 31.14 | 0.56 | 27.25 | 48.96 |
| 3 | 0.071 | 0.64 | 30.49 | 38.67 | 26.27 | 47.84 |
| 4 | 0.057 | 0.63 | 30.03 | 37.26 | 25.55 | 46.95 |
| 5 | 0.029 | 0.57 | 29.67 | 36.13 | 24.92 | 46.18 |
| 6 | 0.0276 | 0.546 | 29.42 | 35.368 | 24.478 | 45.638 |
| 7 | 0.0262 | 0.522 | 29.18 | 34.606 | 24.036 | 45.096 |
| 8 | 0.0248 | 0.498 | 28.93 | 33.844 | 23.594 | 44.554 |
| 9 | 0.0234 | 0.474 | 28.69 | 33.082 | 23.152 | 44.012 |
| 10 | 0.022 | 0.45 | 28.44 | 32.32 | 22.71 | 43.47 |
| 11 | 0.0214 | 0.436 | 28.27 | 31.82 | 22.374 | 43.078 |
| 12 | 0.0208 | 0.422 | 28.1 | 31.32 | 22.038 | 42.686 |
| 13 | 0.0202 | 0.408 | 27.92 | 30.82 | 21.702 | 42.294 |
| 14 | 0.0196 | 0.394 | 27.75 | 30.32 | 21.366 | 41.902 |
| 15 | 0.019 | 0.38 | 27.58 | 29.82 | 21.03 | 41.51 |
| 16 | 0.0184 | 0.37 | 27.44 | 29.426 | 20.758 | 41.2 |
| 17 | 0.0178 | 0.36 | 27.3 | 29.032 | 20.486 | 40.89 |
| 18 | 0.0172 | 0.35 | 27.16 | 28.638 | 20.214 | 40.58 |
| 19 | 0.0166 | 0.34 | 27.02 | 28.244 | 19.942 | 40.27 |
| 20 | 0.016 | 0.33 | 26.88 | 27.85 | 19.67 | 39.96 |
| 21 | 0.0154 | 0.32 | 26.76 | 27.51 | 19.43 | 39.688 |
| 22 | 0.0148 | 0.31 | 26.64 | 27.17 | 19.19 | 39.416 |
| 23 | 0.0142 | 0.32 | 26.51 | 26.83 | 18.95 | 39.144 |
| 24 | 0.0136 | 0.29 | 26.39 | 26.49 | 18.71 | 38.872 |
| 25 | 0.013 | 0.28 | 26.27 | 26.15 | 18.47 | 38.6 |
| 26 | 0.0126 | 0.27 | 26.16 | 25.838 | 18.262 | 38.346 |
| 27 | 0.0122 | 0.26 | 26.05 | 25.526 | 18.054 | 38.092 |
| 28 | 0.0118 | 0.25 | 25.93 | 25.214 | 17.846 | 37.838 |
| 29 | 0.0114 | 0.24 | 25.82 | 24.902 | 17.638 | 37.584 |
| 30 | 0.011 | 0.23 | 25.71 | 24.59 | 17.43 | 37.33 |
| 31 | 0.0106 | 0.224 | 25.61 | 24.306 | 17.236 | 37.09 |
| 32 | 0.0102 | 0.218 | 25.5 | 24.022 | 17.042 | 36.85 |
| 33 | 0.0098 | 0.212 | 25.4 | 23.738 | 16.848 | 36.61 |
| 34 | 0.0094 | 0.206 | 25.29 | 23.454 | 16.654 | 36.37 |
| 35 | 0.009 | 0.2 | 25.19 | 23.17 | 16.46 | 36.13 |
| 36 | 0.0086 | 0.192 | 25.09 | 22.906 | 16.28 | 35.896 |
| 37 | 0.0082 | 0.184 | 25 | 22.642 | 16.1 | 35.662 |
| 38 | 0.0078 | 0.176 | 24.9 | 22.378 | 15.92 | 35.428 |
| 39 | 0.0074 | 0.168 | 24.81 | 22.114 | 15.74 | 35.194 |
| 40 | 0.007 | 0.16 | 24.71 | 21.85 | 15.56 | 34.96 |
| 41 | 0.0068 | 0.152 | 24.61 | 21.582 | 15.396 | 34.73 |
| 42 | 0.0066 | 0.144 | 24.51 | 21.314 | 15.232 | 34.5 |
| 43 | 0.0064 | 0.136 | 24.42 | 21.046 | 15.068 | 34.27 |
| 44 | 0.0062 | 0.128 | 24.32 | 20.778 | 14.904 | 34.04 |
| 45 | 0.006 | 0.12 | 24.22 | 20.51 | 14.74 | 33.81 |
| 46 | 0.0056 | 0.114 | 24.12 | 20.248 | 14.582 | 33.582 |
| 47 | 0.0052 | 0.108 | 24.02 | 19.986 | 14.424 | 33.354 |
| 48 | 0.0048 | 0.102 | 23.92 | 19.724 | 14.266 | 33.126 |
| 49 | 0.0044 | 0.096 | 23.82 | 19.462 | 14.108 | 32.898 |
| 50 | 0.004 | 0.09 | 23.72 | 19.2 | 13.95 | 32.67 |
| 51 | 0.0036 | 0.082 | 23.62 | 18.932 | 13.8 | 32.44 |
| 52 | 0.0032 | 0.074 | 23.52 | 18.664 | 13.65 | 32.21 |
| 53 | 0.0028 | 0.066 | 23.42 | 18.396 | 13.5 | 31.98 |
| 54 | 0.0024 | 0.058 | 23.32 | 18.128 | 13.35 | 31.75 |
| 55 | 0.002 | 0.05 | 23.22 | 17.86 | 13.2 | 31.52 |
| 56 | 0.0018 | 0.044 | 23.11 | 17.602 | 13.054 | 31.284 |
| 57 | 0.0016 | 0.038 | 23.01 | 17.344 | 12.908 | 31.048 |
| 58 | 0.0014 | 0.032 | 22.9 | 17.086 | 12.762 | 30.812 |
| 59 | 0.0012 | 0.026 | 22.8 | 16.828 | 12.616 | 30.576 |
| 60 | 0.001 | 0.02 | 22.69 | 16.57 | 12.47 | 30.34 |
| 61 | 0.0006 | 0.012 | 22.58 | 16.272 | 12.32 | 30.088 |
| 62 | 0.0002 | 0.004 | 22.47 | 15.974 | 12.17 | 29.836 |
| 63 | −0.0002 | −0 | 22.37 | 15.676 | 12.02 | 29.584 |
| 64 | −0.0006 | −0.01 | 22.26 | 15.378 | 11.87 | 29.332 |
| 65 | −0.001 | −0.02 | 22.15 | 15.08 | 11.72 | 29.08 |
| 66 | −0.0014 | −0.03 | 22.03 | 14.778 | 11.572 | 28.808 |
| 67 | −0.0018 | −0.04 | 21.91 | 14.476 | 11.424 | 28.536 |
| 68 | −0.0022 | −0.04 | 21.79 | 14.174 | 11.276 | 28.264 |
| 69 | −0.0026 | −0.05 | 21.67 | 13.872 | 11.128 | 27.992 |
| 70 | −0.003 | −0.06 | 21.55 | 13.57 | 10.98 | 27.72 |
| 71 | −0.0034 | −0.07 | 21.42 | 13.23 | 10.82 | 27.412 |
| 72 | −0.0038 | −0.08 | 21.29 | 12.89 | 10.66 | 27.104 |
| 73 | −0.0042 | −0.08 | 21.15 | 12.55 | 10.5 | 26.796 |
| 74 | −0.0046 | −0.09 | 21.02 | 12.21 | 10.34 | 26.488 |
| 75 | −0.005 | −0.1 | 20.89 | 11.87 | 10.18 | 26.18 |
| 76 | −0.0054 | −0.11 | 20.74 | 11.496 | 10.006 | 25.822 |
| 77 | −0.0058 | −0.12 | 20.59 | 11.122 | 9.832 | 25.464 |
| 78 | −0.0062 | −0.13 | 20.44 | 10.748 | 9.658 | 25.106 |
| 79 | −0.0066 | −0.14 | 20.29 | 10.374 | 9.484 | 24.748 |
| 80 | −0.007 | −0.15 | 20.14 | 10 | 9.31 | 24.39 |
| 81 | −0.0074 | −0.16 | 19.96 | 9.544 | 9.102 | 23.974 |
| 82 | −0.0078 | −0.17 | 19.78 | 9.088 | 8.894 | 23.558 |
| 83 | −0.0082 | −0.18 | 19.61 | 8.632 | 8.686 | 23.142 |
| 84 | −0.0086 | −0.19 | 19.43 | 8.176 | 8.478 | 22.726 |
| 85 | −0.009 | −0.2 | 19.25 | 7.72 | 8.27 | 22.31 |
| 86 | −0.0098 | −0.21 | 19.01 | 7.156 | 8.016 | 21.762 |
| 87 | −0.0106 | −0.23 | 18.77 | 6.592 | 7.762 | 21.214 |
| 88 | −0.0114 | −0.24 | 18.54 | 6.028 | 7.508 | 20.666 |
| 89 | −0.0122 | −0.26 | 18.3 | 5.464 | 7.254 | 20.118 |
| 90 | −0.013 | −0.271 | 18.06 | 4.9 | 7 | 19.57 |
| 91 | −0.014 | −0.29 | 17.68 | 4.01 | 6.606 | 18.66 |
| 92 | −0.015 | −0.31 | 17.3 | 3.124 | 6.212 | 17.75 |
| 93 | −0.016 | −0.34 | 16.92 | 2.236 | 5.818 | 16.84 |
| 94 | −0.017 | −0.36 | 16.54 | 1.348 | 5.424 | 15.93 |
| 95 | −0.018 | −0.38 | 16.16 | 0.46 | 5.03 | 15.02 |
| 96 | −0.019 | −0.62 | 12.34 | −7.608 | 2.05 | 5.416 |
| 97 | −0.021 | −0.85 | 8.516 | −15.68 | −0.93 | −4.188 |
| 98 | −0.024 | −1.09 | 4.694 | −23.74 | −3.91 | −13.792 |
| 99 | −0.028 | −1.32 | 0.872 | −31.81 | −6.89 | −23.396 |
| 100 | −99 | −1.56 | −2.95 | −39.88 | −9.87 | −33 |

CHART 2
Maximize Chart

| Points | CED | BW | WW | YW | YH | SC | CEM | Milk | $EN | IMF | RE |
|---|---|---|---|---|---|---|---|---|---|---|---|
| 100 | 13 | 12.1 | 58 | 105 | 1.1 | 1.57 | 12 | 31 | 21.65 | 0.57 | 0.77 |
| 99 | 12 | 10.66 | 56 | 101 | 1 | 1.41 | 11 | 30 | 19.51 | 0.51 | 0.7 |
| 98 | 11 | 9.22 | 55 | 99 | 0.9 | 1.31 | 11 | 29 | 18.24 | 0.47 | 0.65 |
| 97 | 11 | 7.78 | 54 | 97 | 0.9 | 1.23 | 11 | 29 | 17.32 | 0.44 | 0.62 |
| 96 | 11 | 4.9 | 53 | 96 | 0.8 | 1.17 | 10 | 28 | 16.55 | 0.42 | 0.6 |
| 95 | 10.6 | 4.78 | 52.4 | 95 | 0.78 | 1.132 | 10 | 27.6 | 16.072 | 0.404 | 0.582 |
| 94 | 10.2 | 4.66 | 51.8 | 94 | 0.76 | 1.094 | 10 | 27.2 | 15.594 | 0.388 | 0.564 |
| 93 | 9.8 | 4.54 | 51.2 | 93 | 0.74 | 1.056 | 10 | 26.8 | 15.116 | 0.372 | 0.546 |
| 92 | 9.4 | 4.42 | 50.6 | 92 | 0.72 | 1.018 | 10 | 26.4 | 14.638 | 0.356 | 0.528 |
| 91 | 9 | 4.3 | 50 | 91 | 0.7 | 0.98 | 10 | 26 | 14.16 | 0.34 | 0.51 |
| 90 | 9 | 4.22 | 49.6 | 90.4 | 0.7 | 0.956 | 9.8 | 25.8 | 13.824 | 0.33 | 0.498 |
| 89 | 9 | 4.14 | 49.2 | 89.8 | 0.7 | 0.932 | 9.6 | 25.6 | 13.488 | 0.32 | 0.486 |
| 88 | 9 | 4.06 | 48.8 | 89.2 | 0.7 | 0.908 | 9.4 | 25.4 | 13.152 | 0.31 | 0.474 |
| 87 | 9 | 3.98 | 48.4 | 88.6 | 0.7 | 0.884 | 9.2 | 25.2 | 12.816 | 0.3 | 0.462 |
| 86 | 9 | 3.9 | 48 | 88 | 0.7 | 0.86 | 9 | 25 | 12.48 | 0.29 | 0.45 |
| 85 | 8.8 | 3.84 | 47.8 | 87.6 | 0.68 | 0.84 | 9 | 24.8 | 12.23 | 0.282 | 0.44 |
| 84 | 8.6 | 3.78 | 47.6 | 87.2 | 0.66 | 0.82 | 9 | 24.6 | 11.98 | 0.274 | 0.43 |
| 83 | 8.4 | 3.72 | 47.4 | 86.8 | 0.64 | 0.8 | 9 | 24.4 | 11.73 | 0.266 | 0.42 |
| 82 | 8.2 | 3.66 | 47.2 | 86.4 | 0.62 | 0.78 | 9 | 24.2 | 11.48 | 0.258 | 0.41 |
| 81 | 8 | 3.6 | 47 | 86 | 0.6 | 0.76 | 9 | 24 | 11.23 | 0.25 | 0.4 |
| 80 | 7.8 | 3.54 | 46.8 | 85.6 | 0.60 | 0.742 | 8.8 | 23.8 | 11.016 | 0.244 | 0.392 |
| 79 | 7.6 | 3.48 | 46.6 | 85.2 | 0.60 | 0.724 | 8.6 | 23.6 | 10.802 | 0.238 | 0.384 |
| 78 | 7.4 | 3.42 | 46.4 | 84.8 | 0.60 | 0.706 | 8.4 | 23.4 | 10.588 | 0.232 | 0.376 |
| 77 | 7.2 | 3.36 | 46.2 | 84.4 | 0.60 | 0.688 | 8.2 | 23.2 | 10.374 | 0.226 | 0.368 |
| 76 | 7 | 3.3 | 46 | 84 | 0.6 | 0.67 | 8 | 23 | 10.16 | 0.22 | 0.36 |
| 75 | 7 | 3.26 | 45.6 | 83.6 | 0.58 | 0.656 | 8 | 23 | 9.97 | 0.214 | 0.354 |
| 74 | 7 | 3.22 | 45.2 | 83.2 | 0.56 | 0.642 | 8 | 23 | 9.78 | 0.208 | 0.348 |
| 73 | 7 | 3.18 | 44.8 | 82.8 | 0.54 | 0.628 | 8 | 23 | 9.59 | 0.202 | 0.342 |
| 72 | 7 | 3.14 | 44.4 | 82.4 | 0.52 | 0.614 | 8 | 23 | 9.4 | 0.196 | 0.336 |
| 71 | 7 | 3.1 | 44 | 82 | 0.5 | 0.6 | 8 | 23 | 9.21 | 0.19 | 0.33 |
| 70 | 6.8 | 3.06 | 43.8 | 81.6 | 0.5 | 0.584 | 7.8 | 22.8 | 9.016 | 0.184 | 0.322 |
| 69 | 6.6 | 3.02 | 43.6 | 81.2 | 0.5 | 0.568 | 7.6 | 22.6 | 8.822 | 0.178 | 0.314 |
| 68 | 6.4 | 2.98 | 43.4 | 80.8 | 0.5 | 0.552 | 7.4 | 22.4 | 8.628 | 0.172 | 0.306 |
| 67 | 6.2 | 2.94 | 43.2 | 80.4 | 0.5 | 0.536 | 7.2 | 22.2 | 8.434 | 0.166 | 0.298 |
| 66 | 6 | 2.9 | 43 | 80 | 0.5 | 0.52 | 7 | 22 | 8.24 | 0.16 | 0.29 |
| 65 | 6 | 2.86 | 42.8 | 79.6 | 0.48 | 0.508 | 7 | 21.8 | 8.084 | 0.156 | 0.284 |
| 64 | 6 | 2.82 | 42.6 | 79.2 | 0.46 | 0.496 | 7 | 21.6 | 7.928 | 0.152 | 0.278 |
| 63 | 6 | 2.78 | 42.4 | 78.8 | 0.44 | 0.484 | 7 | 21.4 | 7.772 | 0.148 | 0.272 |
| 62 | 6 | 2.74 | 42.2 | 78.4 | 0.42 | 0.472 | 7 | 21.2 | 7.616 | 0.144 | 0.266 |
| 61 | 6 | 2.7 | 42 | 78 | 0.4 | 0.46 | 7 | 21 | 7.46 | 0.14 | 0.26 |
| 60 | 5.8 | 2.66 | 41.8 | 77.8 | 0.4 | 0.446 | 7 | 21 | 7.304 | 0.136 | 0.254 |
| 59 | 5.6 | 2.62 | 41.6 | 77.6 | 0.4 | 0.432 | 7 | 21 | 7.148 | 0.132 | 0.248 |
| 58 | 5.4 | 2.58 | 41.4 | 77.4 | 0.4 | 0.418 | 7 | 21 | 6.992 | 0.128 | 0.242 |
| 57 | 5.2 | 2.54 | 41.2 | 77.2 | 0.4 | 0.404 | 7 | 21 | 6.836 | 0.124 | 0.236 |
| 56 | 5 | 2.5 | 41 | 77 | 0.4 | 0.39 | 7 | 21 | 6.68 | 0.12 | 0.23 |
| 55 | 5 | 2.46 | 40.8 | 76.6 | 0.4 | 0.378 | 6.82 | 0.8 | 6.52 | 20.116 | 0.224 |
| 54 | 5 | 2.42 | 40.6 | 76.2 | 0.4 | 0.366 | 6.62 | 0.6 | 6.36 | 40.112 | 0.218 |
| 53 | 5 | 2.38 | 40.4 | 75.8 | 0.4 | 0.354 | 6.42 | 0.4 | 6.20 | 60.108 | 0.212 |
| 52 | 5 | 2.34 | 40.2 | 75.4 | 0.4 | 0.342 | 6.22 | 0.2 | 6.04 | 80.104 | 0.206 |
| 51 | 5 | 2.3 | 40 | 75 | 0.4 | 0.33 | 6 | 20 | 5.89 | 0.1 | 0.2 |
| 50 | 4.8 | 2.26 | 39.8 | 74.6 | 0.38 | 0.318 | 6 | 19.8 | 5.738 | 0.096 | 0.196 |
| 49 | 4.6 | 2.22 | 39.6 | 74.2 | 0.36 | 0.306 | 6 | 19.6 | 5.586 | 0.092 | 0.192 |
| 48 | 4.4 | 2.18 | 39.4 | 73.8 | 0.34 | 0.294 | 6 | 19.4 | 5.434 | 0.088 | 0.188 |
| 47 | 4.2 | 2.14 | 39.2 | 73.4 | 0.32 | 0.282 | 6 | 19.2 | 5.282 | 0.084 | 0.184 |
| 46 | 4 | 2.1 | 39 | 73 | 0.3 | 0.27 | 6 | 19 | 5.13 | 0.08 | 0.18 |
| 45 | 4 | 2.06 | 38.8 | 72.8 | 0.3 | 0.258 | 6 | 19 | 4.972 | 0.076 | 0.174 |
| 44 | 4 | 2.02 | 38.6 | 72.6 | 0.3 | 0.246 | 6 | 19 | 4.814 | 0.072 | 0.168 |
| 43 | 4 | 1.98 | 38.4 | 72.4 | 0.3 | 0.234 | 6 | 19 | 4.656 | 0.068 | 0.162 |
| 42 | 4 | 1.94 | 38.2 | 72.2 | 0.3 | 0.222 | 6 | 19 | 4.498 | 0.064 | 0.156 |
| 41 | 4 | 1.9 | 38 | 72 | 0.3 | 0.21 | 6 | 19 | 4.34 | 0.06 | 0.15 |
| 40 | 3.8 | 1.86 | 37.8 | 71.6 | 0.3 | 0.196 | 5.81 | 8.8 | 4.184 | 0.058 | 0.144 |
| 39 | 3.6 | 1.82 | 37.6 | 71.2 | 0.3 | 0.182 | 5.61 | 8.6 | 4.028 | 0.056 | 0.138 |
| 38 | 3.4 | 1.78 | 37.4 | 70.8 | 0.3 | 0.168 | 5.41 | 8.4 | 3.872 | 0.054 | 0.132 |
| 37 | 3.2 | 1.74 | 37.2 | 70.4 | 0.3 | 0.154 | 5.21 | 8.2 | 3.716 | 0.052 | 0.126 |
| 36 | 3 | 1.7 | 37 | 70 | 0.3 | 0.14 | 5 | 18 | 3.56 | 0.05 | 0.12 |
| 35 | 3 | 1.66 | 36.8 | 69.6 | 0.28 | 0.126 | 5 | 17.8 | 3.39 | 0.046 | 0.114 |
| 34 | 3 | 1.62 | 36.6 | 69.2 | 0.26 | 0.112 | 5 | 17.6 | 3.22 | 0.042 | 0.108 |
| 33 | 3 | 1.58 | 36.4 | 68.8 | 0.24 | 0.098 | 5 | 17.4 | 3.05 | 0.038 | 0.102 |
| 32 | 3 | 1.54 | 36.2 | 68.4 | 0.22 | 0.084 | 5 | 17.2 | 2.88 | 0.034 | 0.096 |
| 32 | 3 | 1.S | 36 | 68 | 0.2 | 0.07 | 5 | 17 | 2.71 | 0.03 | 0.09 |
| 30 | 2.8 | 1.44 | 35.8 | 67.6 | 0.2 | 0.056 | 5 | 16.8 | 2.54 | 0.026 | 0.082 |
| 29 | 2.6 | 1.38 | 35.6 | 67.2 | 0.2 | 0.042 | 5 | 16.6 | 2.37 | 0.022 | 0.074 |
| 28 | 2.4 | 1.32 | 35.4 | 66.8 | 0.2 | 0.028 | 5 | 16.4 | 2.2 | 0.018 | 0.066 |
| 27 | 2.2 | 1.26 | 35.2 | 66.4 | 0.2 | 0.014 | 5 | 16.2 | 2.03 | 0.014 | 0.057 |
| 26 | 2 | 1.2 | 35 | 66 | 0.2 | 0 | 5 | 16 | 1.86 | 0.01 | 0.05 |
| 25 | 1.8 | 1.14 | 34.6 | 65.4 | 0.17 | −0.016 | 4.8 | 16 | 1.654 | 0.004 | 0.044 |
| 24 | 1.6 | 1.08 | 34.2 | 64.8 | 0.16 | −0.032 | 4.6 | 16 | 1.448 | −0.002 | 0.037 |
| 23 | 1.4 | 1.02 | 33.8 | 64.2 | 0.14 | −0.048 | 4.4 | 16 | 1.242 | −0.008 | 0.032 |
| 22 | 1.2 | 0.96 | 33.4 | 63.6 | 0.12 | −0.064 | 4.2 | 16 | 1.036 | −0.014 | 0.026 |
| 21 | 1 | 0.9 | 33 | 63 | 0.1 | −0.08 | 4 | 16 | 0.83 | −0.02 | 0.02 |
| 20 | 0.8 | 0.84 | 32.8 | 62.4 | 0.1 | −0.1 | 4 | 15.8 | 0.617 | −0.024 | 0.01 |
| 19 | 0.6 | 0.78 | 32.6 | 61.8 | 0.1 | −0.12 | 4 | 15.6 | 0.406 | −0.028 | 0 |
| 18 | 0.4 | 0.72 | 32.4 | 61.2 | 0.1 | −0.14 | 4 | 15.4 | 0.194 | −0.032 | −0.01 |
| 17 | 0.2 | 0.66 | 32.2 | 60.6 | 0.1 | −0.16 | 4 | 15.2 | −0.017 | −0.036 | −0.02 |
| 16 | 0 | 0.6 | 32 | 60 | 0.1 | −0.18 | 4 | 15 | −0.23 | −0.04 | −0.03 |
| 15 | −0.2 | 0.52 | 31.6 | 59.4 | 0.07 | −0.204 | 3.8 | 14.6 | −0.506 | −0.046 | −0.04 |
| 14 | −0.4 | 0.44 | 31.2 | 58.8 | 0.06 | −0.228 | 3.6 | 14.2 | −0.782 | −0.052 | −0.05 |
| 13 | −0.6 | 0.36 | 30.8 | 58.2 | 0.04 | −0.252 | 3.4 | 13.7 | −1.057 | −0.057 | −0.06 |
| 12 | −0.8 | 0.28 | 30.4 | 57.6 | 0.02 | −0.276 | 3.2 | 13.4 | −1.334 | −0.064 | −0.07 |
| 11 | −1 | 0.2 | 30 | 57 | 0 | −0.3 | 3 | 13 | −1.61 | −0.07 | −0.07 |
| 10 | −1.4 | 0.06 | 29.2 | 55.8 | −0.02 | −0.336 | 2.6 | 12.6 | −2.014 | −0.07 | −0.098 |
| 9 | −1.8 | −0.08 | 28.4 | 54.6 | −0.04 | −0.372 | 2.2 | 12.2 | −2.417 | −0.09 | −0.116 |
| 8 | −2.2 | −0.22 | 27.6 | 53.4 | −0.06 | −0.408 | 1.8 | 11.8 | −2.822 | −0.1 | −0.134 |
| 7 | −2.6 | −0.36 | 26.8 | 52.2 | −0.07 | −0.444 | 1.4 | 11.4 | −3.226 | −0.11 | −0.152 |
| 6 | −3 | −0.5 | 26 | 51 | −0.1 | −0.48 | 1 | 11 | −3.63 | −0.12 | −0.17 |
| 5 | −6.2 | −0.7 | 20 | 39.6 | −0.32 | −0.892 | −1.6 | 7.4 | −6.336 | −0.177 | −0.33 |
| 4 | −9.4 | −0.9 | 14 | 28.2 | −0.54 | −1.304 | −4.2 | 3.8 | −9.042 | −0.236 | −0.49 |
| 3 | −12.6 | −1.2 | 8 | 16.8 | −0.76 | −1.716 | −6.8 | 0.2 | −11.748 | −0.294 | −0.65 |
| 2 | −15.8 | −1.8 | 2 | 5.4 | −0.97 | −2.127 | −9.4 | −3.4 | −14.454 | −0.352 | −0.81 |
| 1 | −19 | −99 | −4 | −6 | −1.2 | −2.54 | −12 | −7 | −17.16 | −0.41 | −0.97 |

| Points | Fat | % RP | $W | $F | $G | $B |
|---|---|---|---|---|---|---|
| 100 | 0.099 | 0.78 | 32.06 | 43.51 | 28.74 | 50.77 |
| 99 | 0.085 | 0.693 | 1.14 | 0.56 | 27.25 | 48.96 |
| 98 | 0.071 | 0.643 | 0.49 | 38.67 | 26.27 | 47.84 |
| 97 | 0.057 | 0.63 | 0.03 | 37.26 | 25.55 | 46.95 |
| 96 | 0.029 | 0.57 | 29.67 | 36.13 | 24.92 | 46.18 |
| 95 | 0.0276 | 0.546 | 29.42 | 35.368 | 24.478 | 45.638 |
| 94 | 0.0262 | 0.522 | 29.18 | 34.606 | 24.036 | 45.096 |
| 93 | 0.0248 | 0.498 | 28.93 | 33.844 | 23.594 | 44.554 |
| 92 | 0.0234 | 0.474 | 28.69 | 33.082 | 23.152 | 44.012 |
| 91 | 0.022 | 0.45 | 28.44 | 32.32 | 22.71 | 43.47 |
| 90 | 0.0214 | 0.436 | 28.27 | 31.82 | 22.374 | 43.078 |
| 89 | 0.0208 | 0.422 | 28.1 | 31.32 | 22.038 | 42.686 |
| 88 | 0.0202 | 0.408 | 27.92 | 30.82 | 21.702 | 42.294 |
| 87 | 0.0196 | 0.394 | 27.75 | 30.32 | 21.366 | 41.902 |
| 86 | 0.019 | 0.38 | 27.58 | 29.82 | 21.03 | 41.51 |
| 85 | 0.0184 | 0.37 | 27.44 | 29.426 | 20.758 | 41.2 |
| 84 | 0.0178 | 0.36 | 27.3 | 29.032 | 20.486 | 40.89 |
| 83 | 0.0172 | 0.352 | 7.162 | 8.638 | 20.214 | 40.58 |
| 82 | 0.0166 | 0.342 | 7.02 | 28.244 | 19.942 | 40.27 |
| 81 | 0.016 | 0.33 | 26.88 | 27.85 | 19.67 | 39.96 |
| 80 | 0.0154 | 0.32 | 26.76 | 27.51 | 19.43 | 39.688 |
| 79 | 0.0148 | 0.31 | 26.64 | 27.17 | 19.19 | 39.416 |
| 78 | 0.0142 | 0.3 | 26.51 | 26.83 | 18.95 | 39.144 |
| 77 | 0.0136 | 0.29 | 26.39 | 26.49 | 18.71 | 38.872 |
| 76 | 0.013 | 0.28 | 26.27 | 26.15 | 18.47 | 38.6 |
| 75 | 0.0126 | 0.27 | 26.16 | 25.838 | 18.262 | 38.346 |
| 74 | 0.0122 | 0.26 | 26.05 | 25.526 | 18.054 | 38.092 |
| 73 | 0.0118 | 0.25 | 25.93 | 25.214 | 17.846 | 37.838 |
| 72 | 0.0114 | 0.24 | 25.82 | 24.902 | 17.638 | 37.584 |
| 71 | 0.011 | 0.23 | 25.71 | 24.59 | 17.43 | 37.33 |
| 70 | 0.0106 | 0.224 | 25.61 | 24.306 | 17.236 | 37.09 |
| 69 | 0.0102 | 0.218 | 25.5 | 24.022 | 17.042 | 36.85 |
| 68 | 0.0098 | 0.212 | 25.4 | 23.738 | 16.848 | 36.61 |
| 67 | 0.0094 | 0.206 | 25.29 | 23.454 | 16.654 | 36.37 |
| 66 | 0.009 | 0.2 | 25.19 | 23.17 | 16.46 | 36.13 |
| 65 | 0.0086 | 0.192 | 25.09 | 22.906 | 16.28 | 35.896 |
| 64 | 0.0082 | 0.184 | 25 | 22.642 | 16.1 | 35.662 |
| 63 | 0.0078 | 0.176 | 24.9 | 22.378 | 15.92 | 35.428 |
| 62 | 0.0074 | 0.168 | 24.81 | 22.114 | 15.74 | 35.194 |
| 61 | 0.007 | 0.16 | 24.71 | 21.85 | 15.56 | 34.96 |
| 60 | 0.0068 | 0.152 | 24.61 | 21.582 | 15.396 | 34.73 |
| 59 | 0.0066 | 0.144 | 24.51 | 21.314 | 15.232 | 34.5 |
| 58 | 0.0064 | 0.136 | 24.42 | 21.046 | 15.068 | 34.27 |
| 57 | 0.0062 | 0.128 | 24.32 | 20.778 | 14.904 | 34.04 |
| 56 | 0.006 | 0.12 | 24.22 | 20.51 | 14.74 | 33.81 |
| 55 | 0.0056 | 0.114 | 24.12 | 20.248 | 14.582 | 33.582 |
| 54 | 0.0052 | 0.108 | 24.02 | 19.986 | 14.424 | 33.354 |
| 53 | 0.0048 | 0.102 | 23.92 | 19.724 | 14.266 | 33.126 |
| 52 | 0.0044 | 0.096 | 23.82 | 19.462 | 14.108 | 32.898 |
| 51 | 0.004 | 0.09 | 23.72 | 19.2 | 13.95 | 32.67 |
| 50 | 0.0036 | 0.082 | 23.62 | 18.932 | 13.8 | 32.44 |
| 49 | 0.0032 | 0.0742 | 3.52 | 18.664 | 13.65 | 32.21 |
| 48 | 0.0028 | 0.0662 | 3.42 | 18.396 | 13.5 | 31.98 |
| 47 | 0.0024 | 0.0582 | 3.32 | 18.128 | 13.35 | 31.75 |
| 46 | 0.002 | 0.05 | 23.221 | 7.86 | 13.2 | 31.52 |
| 45 | 0.0018 | 0.044 | 23.11 | 17.602 | 13.054 | 31.284 |
| 44 | 0.0016 | 0.038 | 23.01 | 17.344 | 12.908 | 31.048 |
| 43 | 0.0014 | 0.032 | 22.9 | 17.086 | 12.762 | 30.812 |
| 42 | 0.0012 | 0.026 | 22.8 | 16.828 | 12.616 | 30.576 |
| 41 | 0.001 | 0.02 | 22.69 | 16.57 | 12.47 | 30.34 |
| 40 | 0.0006 | 0.012 | 22.58 | 16.272 | 12.32 | 30.088 |
| 39 | 0.0002 | 0.004 | 22.47 | 15.974 | 12.17 | 29.836 |
| 38 | −0.0002 | −0 | 22.37 | 15.676 | 12.02 | 29.584 |
| 37 | −0.0006 | −0.01 | 22.26 | 15.378 | 11.87 | 29.332 |
| 36 | −0.001 | −0.02 | 22.15 | 15.08 | 11.72 | 29.08 |
| 35 | −0.0014 | −0.032 | 2.03 | 14.778 | 11.572 | 28.808 |
| 34 | −0.0018 | −0.04 | 21.91 | 14.476 | 11.424 | 28.536 |
| 33 | −0.0022 | −0.04 | 21.79 | 14.174 | 11.276 | 28.264 |
| 32 | −0.0026 | −0.05 | 21.67 | 13.872 | 11.128 | 27.992 |
| 32 | −0.003 | −0.06 | 21.55 | 13.57 | 10.98 | 27.72 |
| 30 | −0.0034 | −0.07 | 21.42 | 13.23 | 10.82 | 27.412 |
| 29 | −0.0038 | −0.08 | 21.29 | 12.89 | 10.66 | 27.104 |
| 28 | −0.0042 | −0.08 | 21.15 | 12.55 | 10.5 | 26.796 |
| 27 | −0.0046 | −0.09 | 21.02 | 12.21 | 10.34 | 26.488 |
| 26 | −0.005 | −0.1 | 20.89 | 11.87 | 10.18 | 26.18 |
| 25 | −0.0054 | −0.11 | 20.74 | 11.496 | 10.006 | 25.822 |
| 24 | −0.0057 | −0.12 | 20.59 | 11.122 | 9.832 | 25.464 |
| 23 | −0.0062 | −0.13 | 20.44 | 10.748 | 9.658 | 25.106 |
| 22 | −0.0066 | −0.14 | 20.29 | 10.374 | 9.484 | 24.748 |
| 21 | −0.007 | −0.15 | 20.14 | 10 | 9.31 | 24.39 |
| 20 | −0.0074 | −0.16 | 19.96 | 9.544 | 9.102 | 23.974 |
| 19 | −0.0078 | −0.17 | 19.78 | 9.088 | 8.894 | 23.558 |
| 18 | −0.0082 | −0.18 | 19.61 | 8.632 | 8.686 | 23.142 |
| 17 | −0.0086 | −0.19 | 19.43 | 8.176 | 8.478 | 22.726 |
| 16 | −0.009 | −0.2 | 19.25 | 7.72 | 8.27 | 22.31 |
| 15 | −0.0098 | −0.21 | 19.01 | 7.156 | 8.016 | 21.762 |
| 14 | −0.0106 | −0.23 | 18.77 | 6.592 | 7.762 | 21.214 |
| 13 | −0.0114 | −0.24 | 18.54 | 6.028 | 7.508 | 20.666 |
| 12 | −0.0122 | −0.26 | 18.3 | 5.464 | 7.254 | 20.118 |
| 11 | −0.013 | −0.27 | 18.06 | 4.9 | 7 | 19.57 |
| 10 | −0.014 | −0.29 | 17.68 | 4.01 | 6.606 | 18.66 |
| 9 | −0.015 | −0.31 | 17.3 | 3.124 | 6.212 | 17.75 |
| 8 | −0.016 | −0.34 | 16.92 | 2.236 | 5.818 | 16.84 |
| 7 | −0.017 | −0.36 | 16.54 | 1.348 | 5.424 | 15.93 |
| 6 | −0.018 | −0.38 | 16.16 | 0.46 | 5.03 | 15.02 |
| 5 | −0.019 | −0.62 | 12.34 | −7.608 | 2.05 | 5.416 |
| 4 | −0.021 | −0.85 | 8.516 | −15.68 | −0.93 | −4.188 |
| 3 | −0.024 | −1.09 | 4.694 | −23.74 | −3.91 | −13.792 |
| 2 | −0.028 | −1.32 | 0.872 | −31.81 | −6.89 | −23.396 |
| 1 | −99 | −1.56 | −2.95 | −39.88 | −9.87 | −33 |

We claim:

1. A method of choosing a bull from a multiplicity of bulls for mating with a cow, the cow and bulls defining a set of potential mating pairs, each animal having a set of EPDs for a set of EPD traits;
selecting a chosen group of EPDs;
choosing a prioritizing emphasis to be placed upon each EPD trait of the chosen group of EPD traits;
choosing a maximizing, minimizing or optimizing option for each EPD trait of the EPD group;
determining a projected offspring EDP for each EPD trait of each cow-bull combination of the set of potential pairs;
converting the projected offspring EDP to a percentage based upon the option chosen from the choosing step;
applying the prioritized emphasis based on the prioritizing of the deciding step to each percentage of the converting step; and
summing the percentages from the applying step for each mating combination; and
selecting the mating combination based upon the summed percentages.

2. A method of choosing a bull from a multiplicity of bulls for mating with a cow to yield offspring, the cow and each bull defining a set of potential mating pairs, and each animal having EPD criteria associated therewith, the method comprising the steps of:
A. Selecting a set of EPDs from the EPD criteria;
B. Prioritizing the EPDs in the selected set of EPDs;
C. Selecting offspring preference values for each EPD;
D. Performing a calculation utilizing the EPD criteria set for each cow-bull combination;
E. Based on the offspring preference values, determining a percentile score for each combined EPD;
F. Modifying each determined percentile score based on the step of prioritizing;
G. Summing the modified percentile scores for each desired cow-bull combination;
H. Selecting a desired cow-bull combination based on the summed percentile scores.

3. The method of claim 2, wherein the step of calculating comprises applying a mathematical formula to the combined EPD criteria.

4. The method of claim 3, wherein the step of calculating comprises determining the average between the combined EPD criteria.

5. The method of claim 2, wherein the step of prioritizing involves assigning a prioritized value to each EPD.

6. The method of claim 5, wherein the prioritized value is a percentage value, the total of all percentage values equaling 100%.

7. The method of claim 2, wherein the selection of offspring preference values comprises the step of categorizing each EPD trait.

8. The method of claim 7, wherein available categories include a maximum or a minimum.

9. The method of claim 8, wherein the selection of offspring preference values comprises assigning an optimum value between the maximum and minimum.

10. A method of choosing a bull from a multiplicity of bulls for mating with a cow to yield offspring, the cow and each bull defining a set of potential mating pairs, the method comprising the steps of:
A. Identifying in the cow a multiplicity of quantifiable characteristics;
B. Assigning an EPD number to each identified quantifiable characteristic of the cow;
C. Identifying in each bull a multiplicity of quantifiable characteristics;
D. Assigning an EPD number to each identified quantifiable characteristic of each bull;
E. Selecting a subset of quantifiable characteristics from those quantifiable characteristics of the cow and bulls for which an EPD number has been assigned;
F. Prioritizing each quantifiable characteristic in the subset;
G. Selecting a maximum, minimum or optimum level for each quantifiable characteristic in the subset;
H. Performing a calculation utilizing the cow EPD number and a first bull's EPD number for a specified quantifiable characteristic to yield an acutal mating calculation value;
I. Repeating Step H for each quantifiable characteristic in the subset;
J. Repeating Steps H-I for each cow-bull combination;
K. Based on the maximum, minimum or optimum level of a mating calculation value, determining a percentile score for each actual mating calculation value;
L. Modifying each determined percentile score based on the step of prioritizing;
M. Summing the modified percentile scores for each desired cow-bull combination;
N. Selecting a desired cow-bull combination based on the summed percentile scores.

11. The method of claim 10, wherein the step of determining a percentile score comprises utilizing the mating calculation value, the selected level and the specified quantifiable characteristic associated with the mating calculation value to determine a percentile score.

12. The method of claim 11, wherein the step of determining a percentile score comprises utilizing the mating calculation value, the selected level and the specified quantifiable characteristic associated with the mating calculation value to determine a percentile score from a look-up table.

13. The method of claim 12, wherein the look-up table is a percentile EPD breed chart.

14. The method of claim 11, wherein the step of determining a percentile score comprises utilizing the mating calculation value, the selected level and the specified quantifiable characteristic associated with the mating calculation value to determine a percentile score from performing a mathematical calculation.

15. The method of claim 11, wherein the selection level for each quantifiable characteristic comprises the step of categorizing each quantifiable characteristic based on its importance in the evaluation process.

16. The method of claim 15, wherein available categories include a maximum level of importance or a minimum level of importance for the quantifiable characteristic.

17. The method of claim 16, wherein the selection level for each quantifiable characteristic comprises assigning a value between the maximum and minimum levels of importance.

18. The method of claim 17, wherein the percentile score is optimized by selecting a desired mating calculation value, obtaining a percentile score associated with the desired mating calculation value; identifying a percentile score for the actual mating calculation value; determining the difference between the optimized percentile score and the actual percentile score; subtracting the determined difference from 100; and utilizing the result as the conversion value or percentile score for the optimized EPD.
